(19) United States
(12) Patent Application Publication
(10) Pub. No.: US 2005/0065171 A1
(43) Pub. Date: Mar. 24, 2005

(54) Substituted purine derivatives

(76) Inventors: William C. Shakespeare, Southborough, MA (US); Chester A. Metcalf III, Needham, MA (US); Tomi K. Sawyer, Southborough, MA (US); Yihan Wang, Newton, MA (US); Rajeswari Sundaramoorthi, Watertown, MA (US); David C. Dalgarno, Brookline, MA (US); Regine Bohacek, Boston, MA (US); Manfred Weigele, Cambridge, MA (US)

Correspondence Address: David L. Berstein;ARIAD Pharmaceuticals, Inc., 26 Landsdowne Street, Cambridge, MA, 02139-4234, US

(21) Appl. No.: 10/876,116

(22) Filed: Jun. 24, 2004

Related U.S. Application Data
(60) Provisional application No. 60/482,259, filed on Jun. 25, 2003.
(60) Provisional application No. 60/485,542, filed on Jul. 8, 2003.
(60) Provisional application No. 60/544,523, filed on Feb. 13, 2004.

(51) Int. Cl. A61K 31/522; C12Q 1/68; C07D 473/12

(52) U.S. Cl. 514/263.22; 514/263.37; 544/276

(57) ABSTRACT

This invention relates to compounds of the general formula: 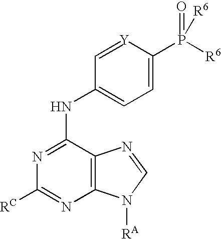
in which Y, R^{A}, R^{C} and R⁶ are as defined herein, and to their preparation and use.

### RELATED APPLICATIONS

The present application claims priority under 35 U.S.C. §119(e) to U.S. Provisional Patent Application No. 60/482,259, filed Jun. 25, 2003, U.S. Provisional Patent Application No. 60/485,542 filed Jul. 8, 2003, and U.S. Provisional Patent Application No. 60/544,523, filed Feb. 13, 2004, the entire contents of each of these applications are hereby incorporated by reference.

### BACKGROUND OF THE INVENTION

The purine ring system has been explored in some detail by academic and industrial researchers, leading to the production and evaluation of a variety of derivatives for various applications. This invention concerns a family of purine derivatives and their use in treating cancers and other diseases, including among others solid tumors and leukemias, including those which are resistant to one or more other therapies.

### DESCRIPTION OF THE INVENTION

1. General Description of Compounds of the Invention

The compounds of this invention have a broad range of useful biological and pharmacological activities, permitting their use in pharmaceutical compositions and treatment methods for treating cancer, including solid tumors and leukemias, especially those mediated by Src or by dysregulation of Abl, including, among others, cases which are resistant or refractory to other treatment. Included are compounds of Formula I: 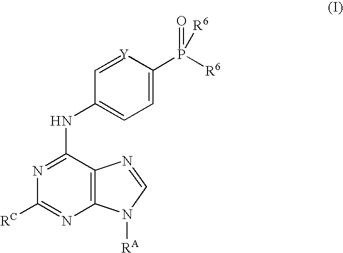
in which Y is CH or N; R^{A} is an aliphatic moiety; R⁶ in each occurrence, is an independently selected aliphatic group of 1-8 carbon atoms, or the two R⁶ moieties may be linked to one another through a carbon-carbon or carbon-heteroatom bond to form a ring, generally of up to 10 members; R^{C} is halogen, R, or -ZR, wherein Z is —O—, —S—, or NR, and each occurrence of R (or R′, R″, etc.) without a further alphanumeric superscript is independently hydrogen or an aliphatic, heteroaliphatic, aryl, or heteroaryl moiety;
wherein in each of the foregoing groups, each aliphatic or heteroaliphatic moiety may be branched or unbranched, cyclic or acyclic, substituted or unsubstituted, and may contain one or more unsaturated bonds, and each aryl and heteroaryl moiety may be substituted or unsubstituted.

The foregoing definitions of R (and R′, R″, etc.), R^{A}, R^{C}, R⁶, Y and Z are further elaborated upon and exemplified below, and as such, apply to all subsequent occurrences except to the extent otherwise specified.

2. Featured Classes of Compounds and their Use, Generally

One class of compounds which is of special interest for use in this invention contains compounds of Formula I, as are described just above in Part 1, in which each R⁶ is an independently chosen C1-C6 (i.e., containing 1-6 carbon atoms) alkyl or alkenyl group, or both R⁶ moieties are covalently linked together to form a ring (e.g., a 3-8-membered ring), in any case, optionally substituted. This class is illustrated by compounds in which —P(═O)R⁶R⁶′ comprises unsubstituted aliphatic groups such as: 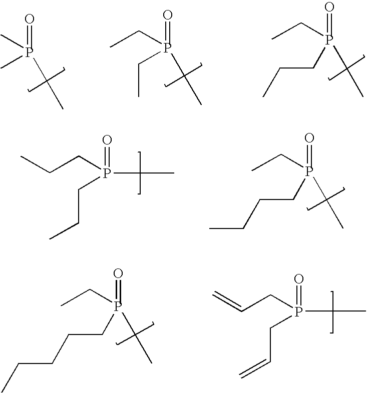
substituted aliphatic groups such as: 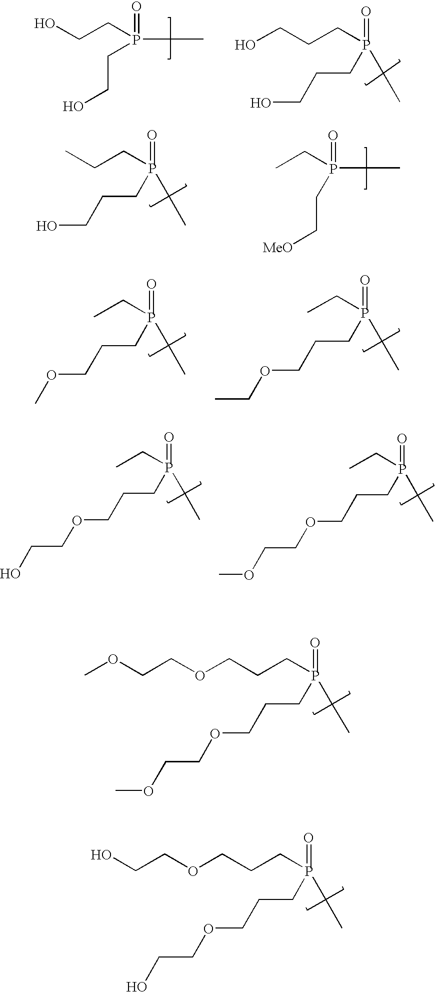
and cyclic groups such as: 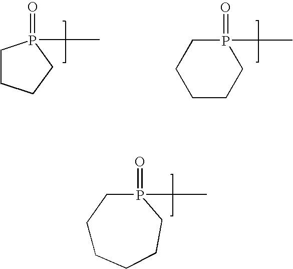
In one subclass, each R⁶ group is a C2-C5 aliphatic group, preferably alkyl or alkenyl, optionally substituted. Compounds of particular interest include those of the following structures: 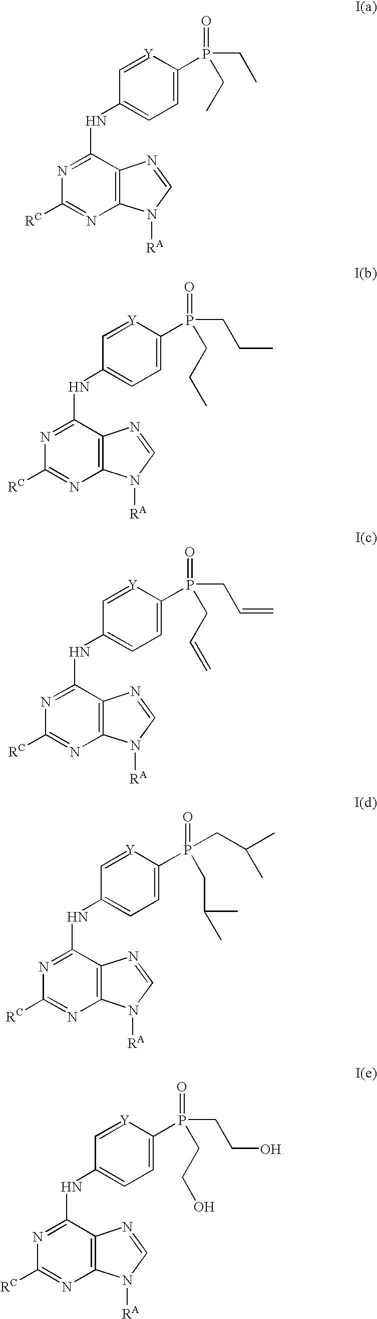
The following structures illustrate several exemplary subsets of this first class. Others will be readily apparent to the reader. 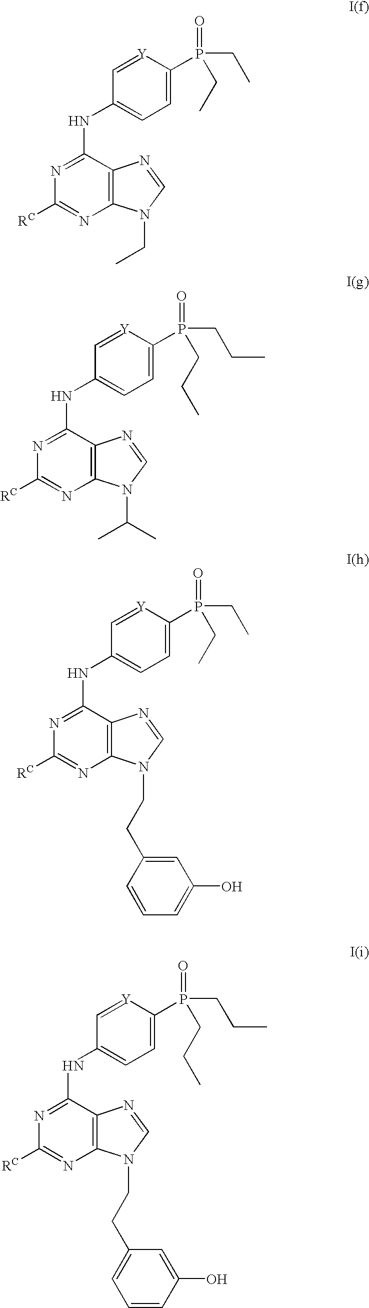 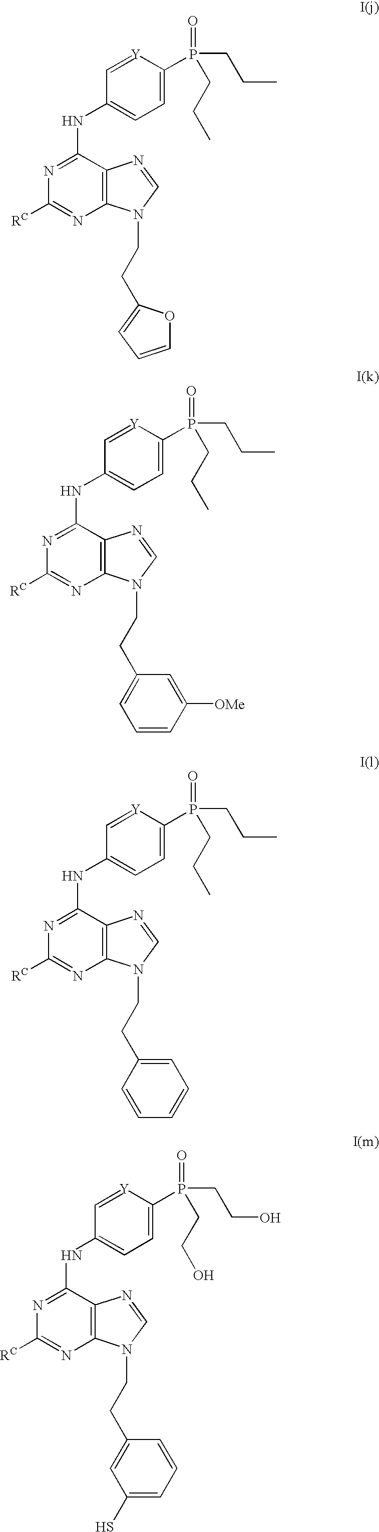 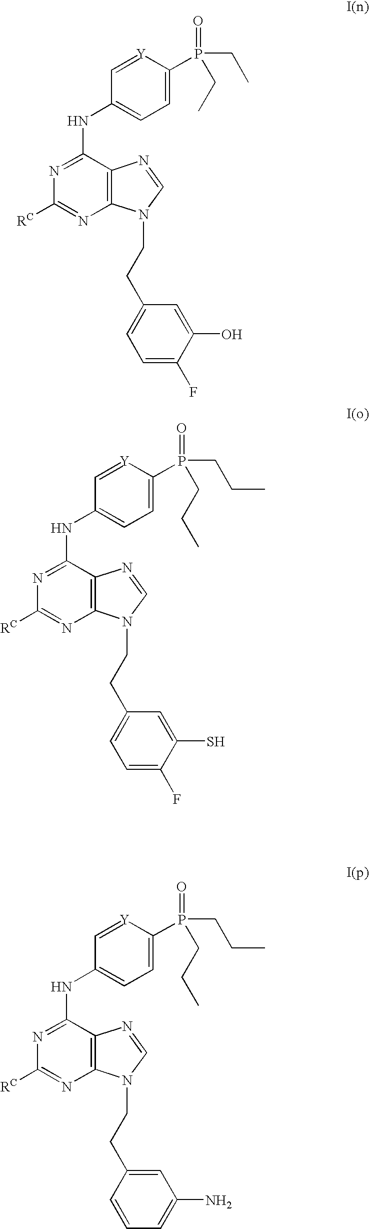 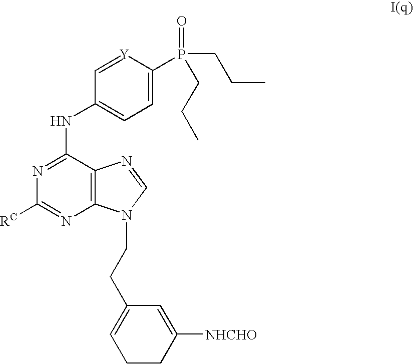
Compounds in which Y is CH, as well as compounds in which Y is N, are both of interest.

A second class of compounds which is of special interest for use in this invention contains compounds of Formula I as are described above in Part 1, including, among others, compounds of any of formulas I(a) through I(g), in which R^{A} is a C2-C6 aliphatic group, substituted with an aryl or heteroaryl moiety which itself may be substituted with one or more optional substituents. This class is illustrated by those of the previously noted structures and substructures having an R^{A} which contains an aliphatic group linked to an aryl or heteroaryl group, as well as by the generalized phenethyl compounds of Formula II: 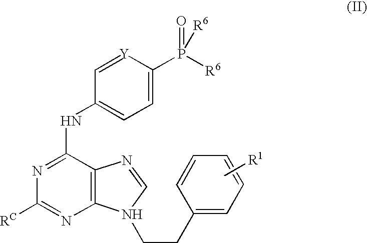
in which R¹ represents one or more optional substituents and the other variable groups are as previously defined. Further illustrations include compounds of Formula II in which the optionally substituted phenethyl moiety is one of the following (in which each “X” is R (preferably a C1-C6 alkyl such as methyl, ethyl, etc., which may be optionally substituted) or halogen, i.e., F, Cl, Br, or I: 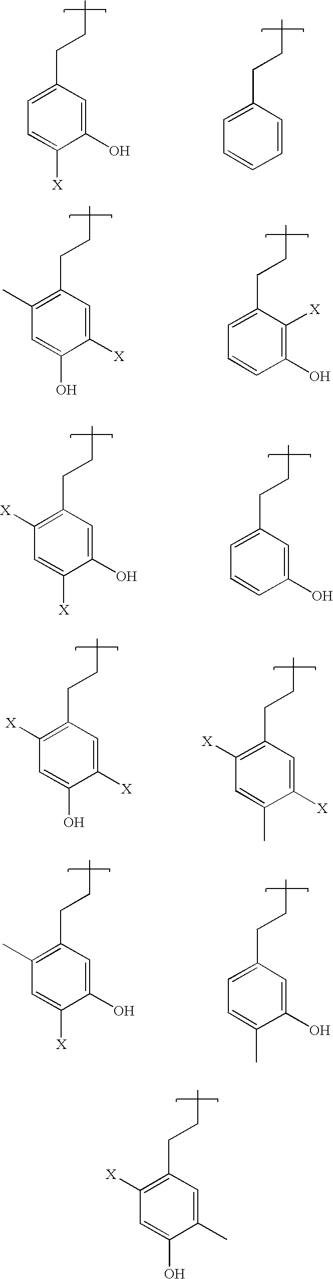
A phenethyl group bearing at least a meta hydroxyl group, or an ether, ester, carbamate, sulfonamide, phosphate, phosphonate or other pharmaceutically acceptable derivative of a meta hydroxyl group, is of particular interest. Phenethyl groups bearing a 3-hydroxy group together with either a 2-methyl, 2-ethyl or other meta-C1-C6 alkyl group or a 2-halo group are also of special interest: 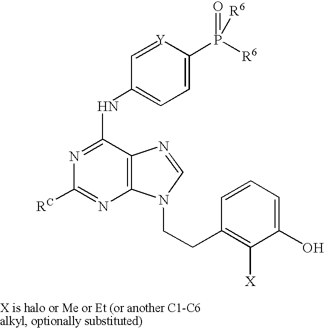
Illustrative subclasses of compounds containing further elaborated hydroxyphenethyl R^{A} moieties which are of particular interest include the following, where the variable groups are as defined previously:
II(a) Phenethyl Ethers: 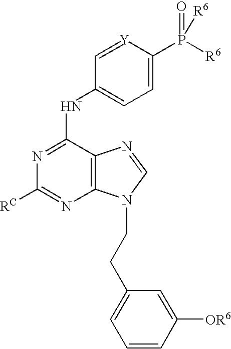
including, for example, ethers of the various C1-C6 alkyl groups (methyl, ethyl, n-propyl, i-propyl, etc.), optionally bearing substituents such as a terminal hydroxy or alkoxy group. Also included are compound s in which R⁶ is —CH₂OC(O)OR⁷, where R⁷ (which may be optionally substituted) is a C1-C6 aliphatic group (methyl, ethyl, n-propyl, i-propyl, etc.), or an aryl or heteroaryl group. Examples of such —OR⁶ groups include —OCH₂OC(O)OMe, —OCH₂OC(O)OEt, —OCH₂OC(O)OCH₂CH₂OMe, —OCH₂OC(O)OBz, etc.
   II(b) Phenethyl Esters: 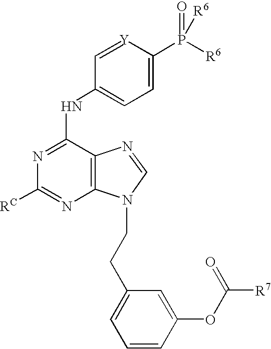
including, for example esters in which R7 is as defined above, including embodiments in which it is an unsubstituted C1-C6 alkyl group, a heterocyclic group, or a C1-C6 alkyl group bearing a hydroxyl, amino or carboxyl substituent. These are illustrated by the following types of derivatives of the hydroxyphenethyl substituents: 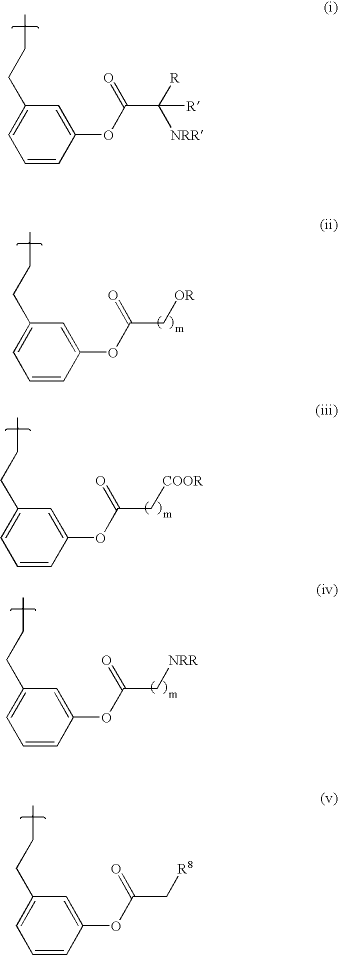
   in which m is an integer from 1 through 6 (in many cases 2-4), R⁸ is a heterocyclic group and the remaining variable groups are as defined elsewhere herein.

Of particular interest are glycinates, i.e., compounds of Formula II(b) in which the hydroxyphenethyl derivative is of type (i), just above, and —NRR′ is —NH₂ or a mono- or di-alkyl amine and the remaining R and R′ are independently H or a C1-C6 alkyl group.

Also of special interest are the hydroxy-, alkoxy-, aryloxy-, heteroaryloxy, carboxy- and amino-substituted derivatives illustrated by structures (ii) through (iv), above. Note that in the case of the amino-substituted compounds of type (iv), the amino group —NRR′ is —NH₂ or is a mono- or di-substituted amine, such as a mono- or dialkyl amine, or is a heterocyclic moiety such as a morpholino-, piperazine- or N-alkyl piperazino-moiety: 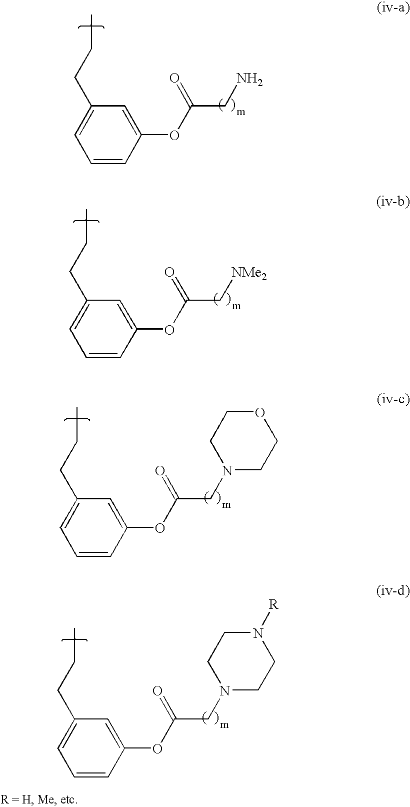
II(c) Phenethyl Carbamates: 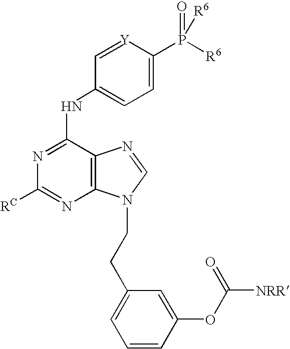
especially those in which —NRR′ is a mono- or di-alkyl amine in which the alkyl group is a substituted or unsubstituted C1-C6 alkyl group or is a heterocyclic moiety such as a morpholino-, piperazine- or N-alkyl piperazino-moiety. Unsubstituted alkyl groups include methyl, ethyl, n-propyl, i-propyl, etc. Examples of substituted alkyl groups include C1-C6 (often C2-C4) alkyl groups bearing substituents such as hydroxyl, alkoxyl, alkoxy-alkoxyl, amino (unsubstituted or mono- or disubstituted), —C(O)NRR′, morpholino, piperzino, N-alklyl piperazino, etc. These are illustrated by compounds having as R^{A} a phenethyl group bearing a carbamate, i.e., —OC(O)NRR′, where each R and R′ is independently H or a group such as Me, Et, iPr, benzyl, substituted benzyl, —CH₂CH₂OH, —CH₂CH₂OMe, —CH₂CH₂OCH₂CH₂OMe, —CH₂CH₂NH₂, —CH₂CH₂NHMe, —CH₂CH₂NMe₂, —CH₂CH₂G (where G is a morpholine, piperizine or N-methylpiperizine group, linked in these cases by the available ring nitrogen atom to the ethyl group, as illustrated in several of the non-limiting examples just below), —CH(Me)CH₂OH, —CH(Me)CH₂OMe, —CH(Me)CH₂OCH₂CH₂OMe, —CH(Me)CH₂NH₂, —CH(Me)CH₂NHMe, —CH(Me)CH₂NMe₂, —CH(Me)CH₂G, —CH₂CONRR′ or —C(Me)₂CH₂CONRR′ (in which R and R′ are independently H or a C1-C6 alkyl or NRR′ is G), or —CH₂C(O)OR or —C(Me)₂CH₂OR or —C(Me)₂CH₂CO₂R (in which R is H or a C1-C6 alkyl). Of special interest are carbamates —OC(O)NRR′, wherein R′ is —CH(R¹⁰)CO₂H (and C1-C6 alkyl esters and amides thereof) in which R¹⁰ is a substituted or unsubstituted C1-C6 alkyl group such that H₂NCH(R¹⁰)CO₂H (or HNCH(R¹⁰)CO₂H) encompasses among others the 20 common amino acids: alanine, cysteine, glycine, isoleucine, leucine, methionine, phenylalanine, proline, tryptophan, tyrosine, valine, arginine, asparagine, aspartic acid, glutamine, glutamic acid, histidine, lysine, serine, and threonine. Other carbamates or interest include those in which —NRR′ comprises hydroxyproline, hydroxyserine, homoserine, homocysteine, orinithine, beta alanine, gamma amino butyric acid, statine, etc. Several non-limiting examples are drawn below: 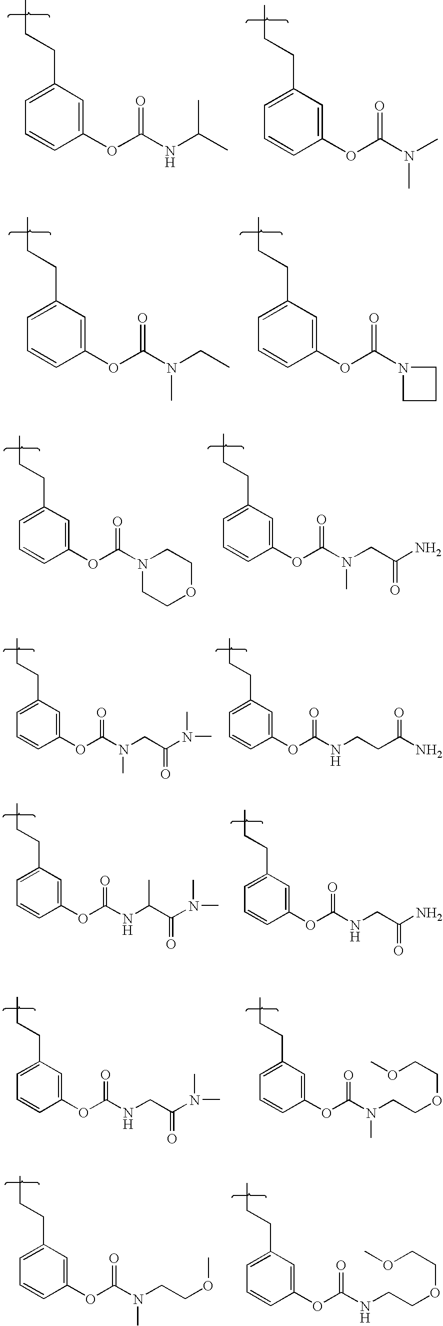 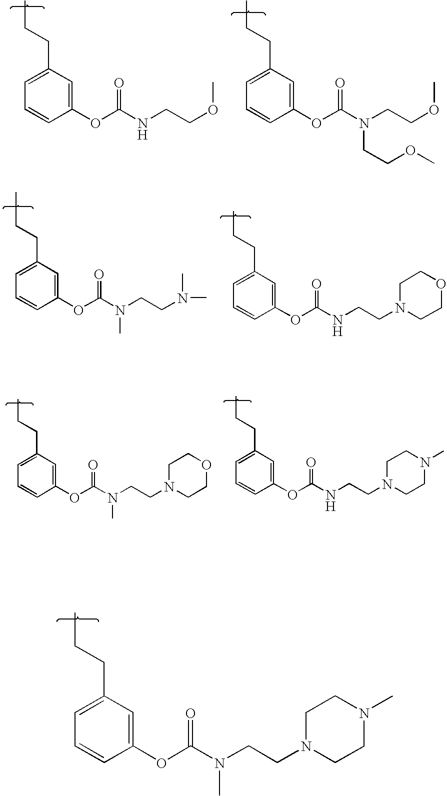
   II(d)-(f) Phenethyl Phosphates, Phosphenates and Sulfonamides: 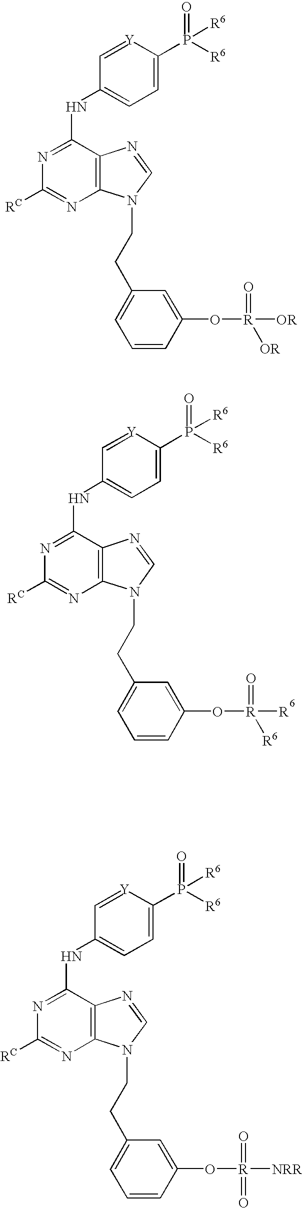
where the variable groups are as previously defined.

Note also in connection with the illustrative subclasses II(a)-II(f), that the phenyl ring of the phenethyl RA group may also bear one or more additional substituents, e.g. a substituent X at ring position 2.

A third class of compounds of special interest are as shown in Formula I and described in Part 1, but in this case, specifically contain an R^{C} moiety which is linked to the purine ring system through a carbon-carbon bond, e.g., when R^{C} is a substituted or unsubstituted aliphatic or aryl group, among others. Compounds of this class include, among others, compounds of Formulas II, II(a) and II(b) including, among others, compounds of any of Formulas I(a) through I(q), and any compounds described in connection with any of the preceding classes, subclasses, subsets or compounds of interest that further contain a carbon-linked R^{C} moiety as just described.

Examples further include compounds of Formula I, Formula II, II(a) and II(b) or any of Formulas I(a) through I(q) in which R^{C} is an alkyl or cycloalkyl moiety which may be optionally substituted. Other examples include compounds of Formula I, Formula II, II(a) and II(b) or any of Formulas I(a) through I(q) in which R^{C} is a heteroaliphatic or heterocyclic moiety which is linked through one of its carbon atoms to the purine ring system. For instance, this class includes compounds of Formula III: 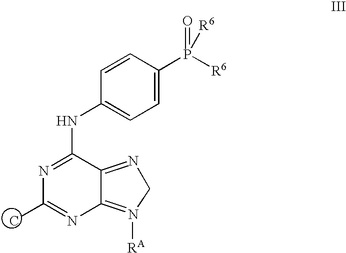
in which Ring “C” is a 3-12-membered ring system linked by one of its carbon atoms to the purine ring.

In one subclass of this third class, the Ring “C” moiety is a cyclic aliphatic group which is unsubstituted or which is substituted with one or more groups limited to hydrocarbon groups (alkyl, alkenyl, phenyl, alkylphenyl, etc.), halogens or halogenated hydrocarbons (haloalkyl, dihaloalkyl, haloalkenyl, halophenyl, etc.)

In another subclass, the Ring “C” moiety is a monocycloalkyl group such as a cyclopropyl, cyclobutyl, cyclopentyl, cyclohexyl, or cycloheptyl ring.

In another subclass, the Ring “C” moiety is a bicycloalkyl group such as norbornane and bicyclo[3.3.1]nonane, among others.

Further illustrating this class are compounds of Formula III containing a ring “C” which comprises a C3-C12 cycloalkyl moiety; each R⁶ comprises a C1-C8 alkyl moiety; and R^{A} comprises a C2-C6 aliphatic group (preferably an ethylene group) optionally containing an aryl or heteroaryl substituent which may itself bear one or more optional substituents. In a subset of such compounds, the compounds contain a ring “C” which comprises a C3-C7 cycloalkyl moiety; each R⁶ comprises a C2-C5 alkyl moiety; and R^{A} comprises a C2-C4 alkyl or alkenyl group or an ethylene group containing an aryl or heteroaryl substituent which may itself bear one or more optional substituents. Of special interest is the subclass of such compounds of Formula III in which R^{A} is a phenethyl group, optionally bearing one or more ring substituents, e.g., as illustrated in connection with the examples noted previously and elsewhere herein (including among others a m-hydroxyphenethyl group, or ester or phosphate derivative thereof as previously described); each R⁶ is C2-C4 alkyl, preferably ethyl or propyl; and Ring “C” is a C3-C7 cycloalkyl moiety. These include compounds of Formula II and of Formulas I(a) through I(q) in which each R⁶ is C2-C4 alkyl, preferably ethyl or propyl, and R^{C} is a C3-C7 cycloalkyl moiety. Also of interest is the subclass in which R^{A} is ethyl or i-propyl.

A fourth class of compounds of special interest are as described above in Part 1 generally, but in this case, contain an R^{C} moiety which is linked to the purine ring system through a carbon-nitrogen bond, e.g., when R^{C} is a substituted or unsubstituted heteroaliphatic, heterocyclic or heteroaryl group, among others. Compounds of this class include, among others, compounds of Formulas II, II(a) or II(b) including, among others, compounds of any of Formulas I(a) through I(q), and any compounds described in connection with any of the preceding classes, subclasses, subsets or compounds of interest that further contain a nitrogen-linked R^{C} moiety as just described.

Examples further include compounds of Formula I; Formula II, II(a) or II(b); or any of Formulas I(a) through I(q) in which R^{C} is an amine which is mono- or disubstituted with an aliphatic (i.e., alkyl, alkenyl or alkynyl, or cyclic forms thereof), aryl, heteroaliphatic, heterocyclic or heteroaryl group (which may be substituted or unsubstituted); or R^{C} is a nitrogen-containing heterocyclic moiety which may be optionally substituted, and is linked through a nitrogen atom to the purine ring system. For instance, this class is illustrated by compounds in which R^{C} is one of the following non-limiting examples:

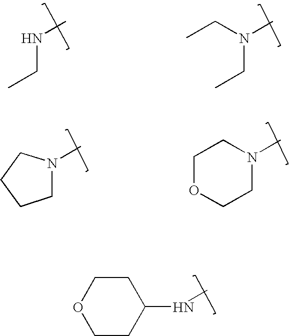

Compounds of this invention which are of greatest current interest have sub-nanomolar IC50 values against Src kinase and Abl kinase and IC50 values against the K-562 or another human CML cell line of 50 nanomolar or better, preferably 25 nM or better, and more preferably 15 nM or better. Some compounds described herein are also active inhibitors of other kinases such as PDGF, c-kit, raf, etc.

Compounds of particular interest include those with one or more of the following characteristics:
have a molecular weight (not including the weight of any counter-ion in the case of a salt or of a moiety added to form a prodrug or other pharmaceutically acceptable derivative) of less than 1000, preferably less than 800 and more preferably less than 700 mass units; or
inhibit Src or Abl kinase or another kinase of interest with an IC50 value of 100 nM or less (as determined using any scientifically acceptable kinase inhibition assay); or
inhibit a given kinase with an IC50 value at least 100-fold lower than their IC50 values for other kinases of interest; or
exibit a cytotoxic or growth inhibitory effect on cancer cell lines maintained in vitro, or in animal studies using a scientifically acceptable cancer cell xenograft model, (especially preferred are compounds of the invention which inhibit proliferation of cultured K562 cells with a potency at least as great as Gleevec, preferably with a potency at least twice that of Gleevec, and more preferably with a potency at least 10 times that of Gleevec as determined by comparative studies.); or
have a structure in which R^{A} is an ethyl group; or
have a structure in which R^{A} is a phenethyl group containing one or more substituents on the phenyl ring thereof; or
have a structure in which Y is CH.

Also provided is a composition comprising at least one of the disclosed compounds or a pharma-ceutically acceptable derivative thereof and at least one pharmaceutically acceptable excipient or additive. Such compositions can be administered to a subject in need thereof to inhibit the growth, development and/or metastasis of cancers, including solid tumors (e.g., breast, colon, pancreatic, CNS and head and neck cancers, among others) and various forms of leukemia, including leukemias and other cancers which are resistant to other treatment, including those which are resistant to treatment with Gleevec or another kinase inhibitor, and generally for the treatment and prophylaxis of diseases or undesirable conditions mediated by one or more kinases which are inhibited by a compound of this invention.

The treatment method of this invention involves administering (as a monotherapy or in combination with one or more other anti-cancer agents, one or more agents for ameliorating side effects, radiation, etc) a therapeutically effective amount of a compound of the invention to a human or animal in need of it in order to inhibit, slow or reverse the growth, development or spread of cancer, including solid tumors or other forms of cancer such as leukemias, in the recipient. Such administration constitutes a method for the treatment or prophylaxis of diseases mediated by one or more kinases inhibited by one of the disclosed compounds or a pharmaceutically acceptable derivative thereof. “Administration” of a compound of this invention encompasses the delivery to a recipient of a compound of the sort described herein, or a prodrug or other pharmaceutically acceptable derivative thereof, using any suitable formulation or route of administration, as discussed herein. Typically the compound is administered one or more times per week, e.g. daily, every other day, 5 days/week, etc. Oral and intravenous administration are of particular current interest.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue (MW>300) thereof. Pharmaceutically acceptable derivatives thus include among others pro-drugs. A pro-drug is a derivative of a compound, usually with significantly reduced pharmacological activity, which contains an additional moiety which is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention.

Particularly favored derivatives and prodrugs of a parent compound are those derivatives and prodrugs that increase the bioavailability of the compound when administered to a mammal (e.g., by permitting enhanced absorption into the blood following oral administration) or which enhance delivery to a biological compartment of interest (e.g., the brain or lymphatic system) relative to the parent compound. Preferred prodrugs include derivatives of a compound of this invention with enhanced aqueous solubility or active transport through the gut membrane, relative to the parent compound.

Thus an important aspect of this invention is a method for treating cancer in a subject in need thereof, which comprises administering to the subject a treatment effective amount of a composition containing a compound of this invention. Various cancers which may be thus treated are noted elsewhere herein and include, among others, cancers which are or have become resistant to another anticancer agent such as Gleevec, Iressa, Tarceva or one of the other agents noted herein. Treatment may be provided in combination with one or more other cancer therapies, include surgery, radiotherapy (e.g., gamma-radiation, neutron beam radiotherapy, electron beam radiotherapy, proton therapy, brachytherapy, and systemic radioactive isotopes, etc.), endocrine therapy, biologic response modifiers (e.g., interferons, interleukins, and tumor necrosis factor (TNF) to name a few), hyperthermia, cryotherapy, agents to attenuate any adverse effects (e.g., antiemetics), and other cancer chemotherapeutic drugs. The other agent(s) may be administered using a formulation, route of administration and dosing schedule the same or different from that used with the compound of this invention.

Such other drugs include but not limited to one or more of the following: an anti-cancer alkylating or intercalating agent (e.g., mechlorethamine, chlorambucil, Cyclophosphamide, Melphalan, and Ifosfamide); antimetabolite (e.g., Methotrexate); purine antagonist or pyrimidine antagonist (e.g., 6-Mercaptopurine, 5-Fluorouracil, Cytarabile, and Gemcitabine); spindle poison (e.g., Vinblastine, Vincristine, Vinorelbine and Paclitaxel); podophyllotoxin (e.g., Etoposide, Irinotecan, Topotecan); antibiotic (e.g., Doxorubicin, Bleomycin and Mitomycin); nitrosourea (e.g., Carmustine, Lomustine); inorganic ion (e.g., Cisplatin, Carboplatin, Oxaliplatin or oxiplatin); enzyme (e.g., Asparaginase); hormone (e.g., Tamoxifen, Leuprolide, Flutamide and Megestrol); mTOR inhibitor (e.g., rapamycin, CCI779, RAD001, compounds disclosed in PCT/US03/03030 or U.S. Ser. No. 10/357,152, etc.); proteasome inhibitor (such as Velcade, another proteasome inhibitor (see e.g., WO 02/096933) or another NF-kB inhibitor, including, e.g., an IkK inhibitor); other kinase inhibitors (e.g., an inhibitor of Src, BRC/Abl, kdr, flt3, aurora-2, glycogen synthase kinase 3 (“GSK-3”), EGF-R kinase (e.g., Iressa, Tarceva, etc.), VEGF-R kinase, PDGF-R kinase, etc); an antibody, soluble receptor or other receptor antagonist against a receptor or hormone implicated in a cancer (including receptors such as EGFR, ErbB2, VEGFR, PDGFR, and IGF-R; and agents such as Herceptin, Avastin, Erbitux, etc.); etc. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference. Examples of other therapeutic agents are noted elsewhere herein and include among others, Zyloprim, alemtuzmab, altretamine, amifostine, nastrozole, antibodies against prostate-specific membrane antigen (such as MLN-591, MLN591RL and MLN2704), arsenic trioxide, bexarotene, bleomycin, busulfan, capecitabine, Gliadel Wafer, celecoxib, chlorambucil, cisplatin-epinephrine gel, cladribine, cytarabine liposomal, daunorubicin liposomal, daunorubicin, daunomycin, dexrazoxane, docetaxel, doxorubicin, Elliott's B Solution, epirubicin, estramustine, etoposide phosphate, etoposide, exemestane, fludarabine, 5-FU, fulvestrant, gemcitabine, gemtuzumab-ozogamicin, goserelin acetate, hydroxyurea, idarubicin, idarubicin, Idamycin, ifosfamide, imatinib mesylate, irinotecan (or other topoisomerase inhibitor, including antibodies such as MLN576 (XR11576)), letrozole, leucovorin, leucovorin levamisole, liposomal daunorubicin, melphalan, L-PAM, mesna, methotrexate, methoxsalen, mitomycin C, mitoxantrone, MLN518 or MLN608 (or other inhibitors of the flt-3 receptor tyrosine kinase, PDFG-R or c-kit), itoxantrone, paclitaxel, Pegademase, pentostatin, porfimer sodium, Rituximab (RITUXAN®), talc, tamoxifen, temozolamide, teniposide, VM-26, topotecan, toremifene, 2C4 (or other antibody which interferes with HER2-mediated signaling), tretinoin, ATRA, valrubicin, vinorelbine, or pamidronate, zoledronate or another bisphosphonate.

This invention further comprises the preparation of a compound of Formula I, Formula II or of any of Formulas I(a) through I(q) or of any other of the compounds of this invention.

The invention also comprises the use of a compound of the invention, or a pharmaceutically acceptable derivative thereof, in the manufacture of a medicament for the treatment either acutely or chronically of cancer (including leukemias and solid tumors, primary or metastatic, including cancers such as noted elsewhere herein and including cancers which are resistant or refractory to one or more other therapies). The compounds of this invention are useful in the manufacture of an anti-cancer medicament. The compounds of the present invention are also useful in the manufacture of a medicament to attenuate or prevent disorders through inhibition of one or more kinases such as Src and abl.

This invention further encompasses a composition comprising a compound of the invention, including a compound of any of the described classes or subclasses, including those of any of Formula I, II or I(a) through (p) among others, preferably in a therapeutically-effective amount, in association with a least one pharmaceutically acceptable carrier, adjuvant or diluent.

Compounds of this invention are also useful as standards and reagents for characterizing various kinases, especially but not limited to Src family kinases, as well as for studying the role of such kinases in biological and pathological phenomena; for studying intracellular signal transduction pathways mediated by such kinases, for the comparative evaluation of new kinase inhibitors; and for studying various cancers in cell lines and animal models.

3. Definitions

In reading this document, the following information and definitions apply unless otherwise indicated. In addition, unless otherwise indicated, all occurrences of a functional group are independently chosen, as the reader is in some cases reminded by the use of a slash mark or prime to indicate simply that the two occurrences may be the same or different (e.g., R, R′, R″, etc.).

The term “aliphatic” as used herein includes both saturated and unsaturated (but non-aromatic), straight chain (i.e., unbranched), branched, cyclic, or polycyclic non-aromatic hydrocarbon moieties, which are optionally substituted with one or more functional groups. Unless otherwise specified, alkyl, other aliphatic, alkoxy and acyl groups preferably contain 1-8 (i.e., “C1-C8”), and in many cases 1-6 (i.e., “C1-C6”), contiguous aliphatic carbon atoms. Illustrative aliphatic groups thus include, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents.

The term “aliphatic” is thus intended to include alkyl, alkenyl, alkynyl, cycloalkyl, and cycloalkenyl moieties.

As used herein, the term “alkyl” includes both straight, branched and cyclic alkyl groups. An analogous convention applies to other generic terms such as “alkenyl”, “alkynyl” and the like. Furthermore, “alkyl”, “alkenyl”, “alkynyl” and like groups may be substituted or unsubstituted.

“Alkyl” refers to groups usually having one to eight, preferably one to six carbon atoms. For example, “alkyl” may refer to methyl, ethyl, n-propyl, isopropyl, cyclopropyl, butyl, isobutyl, sec-butyl, tert-butyl, cyclobutyl, pentyl, isopentyl tert-pentyl, cyclopentyl, hexyl, isohexyl, cyclohexyl, etc. Illustrative substituted alkyl groups include, but are not limited to, fluoromethyl, difluoromethyl, trifluoromethyl, 2-fluoroethyl, 3-fluoropropyl, hydroxymethyl, 2-hydroxyethyl, 3-hydroxypropyl, benzyl, substituted benzyl, phenethyl, substituted phenethyl, etc.

The term “alkenyl” refers to groups usually having two to eight, often two to six carbon atoms. For example, “alkenyl” may refer to prop-2-enyl, but-2-enyl, but-3-enyl, 2-methylprop-2-enyl, hex-2-enyl, hex-5-enyl, 2,3-dimethylbut-2-enyl, and the like. The term “alkynyl,” which also refers to groups having two to eight, preferably two to six carbons, includes, but is not limited to, prop-2-ynyl, but-2-ynyl, but-3-ynyl, pent-2-ynyl, 3-methylpent-4-ynyl, hex-2-ynyl, hex-5-ynyl, etc.

The term “cycloalkyl” as used herein refers specifically to groups having three to 12, preferably three to ten, carbon atoms in a mono-, di- or polycyclic (i.e., ring) structure. Illustrative cycloalkyls include, but are not limited to, cyclopropyl, cyclobutyl, cyclopentyl, cyclohexyl, cycloheptyl, norbornyl and the like, which, as in the case of other aliphatic or heteroaliphatic or heterocyclic moieties, may optionally be substituted.

The term “heteroaliphatic” as used herein refers to aliphatic moieties which contain one or more oxygen, sulfur, nitrogen, phosphorous or silicon atoms, e.g., in place of carbon atoms. Heteroaliphatic moieties may be branched, unbranched or cyclic and include heterocycles such as morpholino, pyrrolidinyl, etc.

“Heterocycle”, “heterocyclyl”, or “heterocyclic” as used herein refers to non-aromatic ring systems having five to fourteen ring atoms, preferably five to ten, in which one or more ring carbons, preferably one to four, are each replaced by a heteroatom such as N, O, or S. Non-limiting examples of heterocyclic rings include 3-1H-benzimidazol-2-one, (1-substituted)-2-oxo-benzimidazol-3-yl, 2-tetrahydrofuranyl, 3-tetrahydrofuranyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

The term “aryl” used alone or as part of a larger moiety as in “aralkyl”, “aralkoxy”, or “aryloxyalkyl”, refers to aromatic ring groups having six to fourteen members, such as phenyl, 1-naphthyl, 2-naphthyl, 1-anthracyl and 2-anthracyl. An “aryl” ring may contain one or more substituents. The term “aryl” may be used interchangeably with the term “aryl ring”. “Aryl” also includes fused polycyclic aromatic ring systems in which an aromatic ring is fused to one or more rings. Non-limiting examples of useful aryl ring groups include phenyl, hydroxyphenyl, halophenyl, alkoxyphenyl, dialkoxyphenyl, trialkoxyphenyl, alkylenedioxyphenyl, naphthyl, phenanthryl, anthryl, phenanthro and the like, as well as 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in a indanyl, phenanthridinyl, or tetrahydronaphthyl, where the radical or point of attachment is on the aromatic ring.

The term “heteroaryl” as used herein refers to stable heterocyclic, and polyheterocyclic aromatic moieties having 3-14 carbon atoms (and usually 5-14 ring atoms), which moieties may be substituted or unsubstituted and may comprise one or more rings. Possible substituents include, among others, any of the previously mentioned substituents. Examples of typical heteroaryl rings include 5-membered monocyclic ring groups such as thienyl, pyrrolyl, imidazolyl, pyrazolyl, furyl, isothiazolyl, furazanyl, isoxazolyl, thiazolyl and the like; 6-membered monocyclic groups such as pyridyl, pyrazinyl, pyrimidinyl, pyridazinyl, triazinyl and the like; and polycyclic heterocyclic ring groups such as benzo[b]thienyl, naphtho[2,3-b]thienyl, thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxalinyl, quinazolinyl, benzothiazole, benzimidazole, tetrahydroquinoline cinnolinyl, pteridinyl, carbazolyl, beta-carbolinyl, phenanthridinyl, acridinyl, perimidinyl, phenanthrolinyl, phenazinyl, isothiazolyl, phenothiazinyl, phenoxazinyl, and the like (see e.g. Katritzky, Handbook of Heterocyclic Chemistry). Further specific examples of heteroaryl rings include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, or benzoisoxazolyl. Heteroaryl groups further include a group in which a heteroaromatic ring is fused to one or more aromatic or nonaromatic rings where the radical or point of attachment is on the heteroaromatic ring. Examples include tetrahydroquinoline, tetrahydroisoquinoline, and pyrido[3,4-d]pyrimidinyl. The term “heteroaryl” also refers to rings that are optionally substituted. The term “heteroaryl” may be used interchangeably with the term “heteroaryl ring” or the term “heteroaromatic”.

An aryl group (including the aryl portion of an aralkyl, aralkoxy, or aryloxyalkyl moiety and the like) or heteroaryl group (including the heteroaryl portion of a heteroaralkyl or heteroarylalkoxy moiety and the like) may contain one or more substituents. Examples of suitable substituents on the unsaturated carbon atom of an aryl or heteroaryl group include halogen or —YR, wherein each occurrence of Y is independently —O—, —S—, —NR—, or a chemical bond; —YR thus encompasses —R, —OR, —SR and —NRR′. Other possible substituents include —Y—C(═O)R, —Y—C(═O)OR, —Y—C(═O)NRR′, —Y—C(═NR)NR′R″, —COCOR, —COMCOR (where M is a bond or a 1-4 carbon aliphatic group), —P(═O)(YR)(Y′R′), —CN, —S(═O)R, —SO₂R, —SO₂NRR′, —NO₂, —NRSO₂R′ and —NRSO₂NR′R″. To illustrate further, substituents in which Y is —NR thus include among others, —NRC(═O)R′, —NRC(═O)NR′, —NRC(═O)OR′, and —NRC(═NH)NR′. Note that R substituents may themselves be substituted or unsubstituted (e.g. non-limiting illustrations of an R moiety include -alkylhalo such as chloromethyl or trichloromethyl; -alkoxyalkyl such as methoxyethyl-; mono-, di- and tri-alkoxyphenyl; methylenedioxyphenyl or ethylenedioxyphenyl; halophenyl; and alkylamino). Additional illustrative examples include 1,2-methylene-dioxy, 1,2-ethylenedioxy, protected OH (such as acyloxy)), phenyl, substituted phenyl, —O-phenyl, —O-(substituted) phenyl, -benzyl, substituted benzyl, —O-phenethyl (i.e., —OCH₂CH₂C₆H₅), —O-(substituted)phenethyl, —C(O)CH₂C(O)R, —CO₂R, —C(═O)R (i.e., acyl in cases in which R is aliphatic, aroyl in cases in which R is aryl and heteroaroyl in cases in which R is heteroaryl), —C(═O)NRR′, —OC(═O)NRR′, —C(═NH)NRR′, and —OC(═NH)NRR′. Further examples of substituents include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl groups.

An aliphatic, heteroaliphatic or non-aromatic heterocyclic group may also contain one or more substituents. Examples of suitable substituents on such groups include those listed above for the carbon atoms of an aryl or heteroaryl group and in addition include the following substituents for a saturated carbon atom: ═O, ═S, ═NR, ═NNRR′, ═NNHC(O)R, ═NNHCO₂R, or ═NNHSO₂R. Illustrative examples of substituents on an aliphatic, heteroaliphatic or heterocyclic group include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl groups.

Illustrative substituents on the nitrogen of an aromatic or non-aromatic heterocyclic ring include —R, —NRR′, —C(═O)R, —C(═O)OR, —C(═O)NRR′, —C(═NR)NR′R″, —COCOR, —COMCOR, —CN, —NRSO₂R′ and —NRSO₂NR′R″.

Examples of substituents on the aliphatic group or the phenyl ring include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl.

This invention encompasses only those combinations of substituents and variables that result in a stable or chemically feasible compound. A stable compound or chemically feasible compound is one that has stability sufficient to permit its preparation and detection. Preferred compounds of this invention are sufficiently stable that they are not substantially altered when kept at a temperature of 40° C. or less, in the absence of moisture or other chemically reactive conditions, for at least a week.

Certain compounds of this invention may exist in tautomeric forms, and this invention includes all such tautomeric forms of those compounds unless otherwise specified.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Thus, this invention encompasses each diasteriomer or enantiomer substantially free of other isomers (>90%, and preferably >95%, free from other stereoisomers on a molar basis) as well as a mixture of such isomers.

Particular optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound.

The optically active compounds of the invention can likewise be obtained by using active starting materials. These isomers may be in the form of a free acid, a free base, an ester or a salt.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a 13 C- or 14 C-enriched carbon are within the scope of this invention.

4. Synthetic Overview

The practitioner has a well-established literature of purine chemistry to draw upon, in combination with the information contained in the examples which follow, for guidance on synthetic strategies, protecting groups, and other materials and methods useful for the synthesis of the compounds of this invention, including compounds containing the various R^{A}, R^{C} and R⁶ substituents. The following references, and the references cited therein, may be of particular interest: U.S. Pat. Nos. 5,365,886; 5,434,150; 5,565,566; 5,869,468; 6,057,305; 5,444,068; 5,635,525; 5,866,702; 5,962,479; 6,057,326; 5,994,361; 6,110,923; 6,028,076; 6,084,095; and 6,107,300; WO 01/44259, 00/43394, 90/09178, 00/44750, 97/49689, 95/35297, 95/19774, 97/35539, 97/16452, 00/49018, 97/20842, 98/16528, 99/07705, 99/62908 and 00/55161; and EP 155911, 478292, 531597, 853084, 454427, 778277, 773023, and 882727.

Various synthetic approaches may be used to produce the compounds described herein, including those approaches depicted schematically below. The practitioner will appreciate that protecting groups may be used in these approaches. “Protecting groups”, are moieties that are used to termporarily block chemical reaction at a potentially reactive site (e.g., an amine, hydroxy, thiol, aldehyde, etc.) so that a reaction can be carried out selectively at another site in a multifunctional compound. In preferred embodiments, a protecting group reacts selectively in good yield to give a protected substrate that is suitable for the planned reactions; the protecting group should be selectively removable in good yield by readily available, preferably nontoxic reagents that do not unduly attack the other functional groups present; the protecting group preferably forms an readily separable derivative (more preferably without the generation of new stereogenic centers); and the protecting group preferably has a minimum of additional functionality to avoid the complication of further sites of reaction. A wide variety of protecting groups and strategies, reagents and conditions for deploying and removing them are known in the art. See, e.g., “Protective Groups in Organic Synthesis” Third Ed. Greene, T. W. and Wuts, P. G., Eds., John Wiley & Sons, New York: 1999. For additional background information on protecting group methodologies (materials, methods and strategies for protection and deprotection) and other synthetic chemistry transformations useful in producing the compounds described herein, see in R. Larock, Comprehensive organic Transformations, VCH Publishers (1989); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995). The entire contents of these references are hereby incorporated by reference.

Amino acid esters, e.g., dimethyl glycinate esters, may be prepared by reacting the hydroxyl compound with the amino acid (e.g., N,N-dimethyl glycine) using EDCI/DMAP/DMA using conventional conditions. Alternatively, an amino acid ester may be prepared by converting the hydroxy compound to the corresponding chloro ester, e.g. using ClCH₂(C═O)Cl, and then displacing the chloro group with the desired amine, e.g., dimethylamine in DMF.

A hydroxy compound may be phosphorylated by reaction with (i-Pr)₂N—P(OtBu)₂ and then MCPBA to form the di-t-butyl phosphonate ester, followed by reaction with TFA to remove the t-butyl groups.

In addition, one may chose reagents enriched for a desired isotope, e.g. deuterium in place of hydrogen, to create compounds of this invention containing such isotope(s). Compounds containing deuterium in place of hydrogen in one or more locations, or containing various isotopes of C, N, P and O, are encompassed by this invention and may be used, for instance, for studying metabolism and/or tissue distribution of the compounds or to alter the rate or path of metabolism or other aspects of biological functioning.

Two general, overall synthetic approaches are illustrated below:

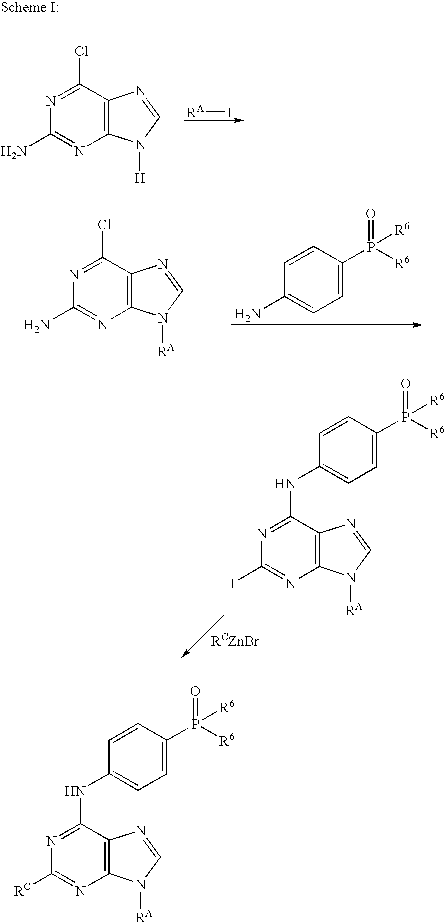

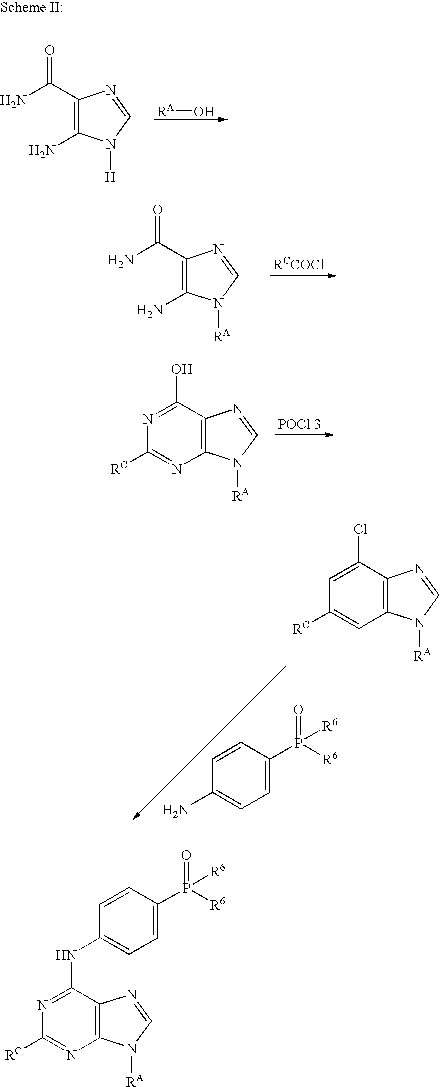

Accordingly, intermediates such as the following: 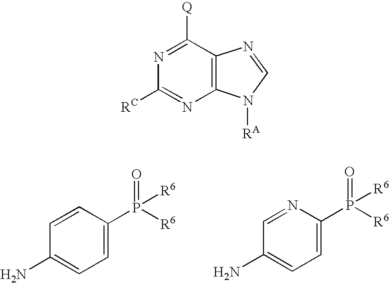
are of considerable utility, where R^{A}, R^{C} and R⁶ are as defined previously. Those intermediates of interest include, among others, compounds containing the various combinations of R^{A} and R^{C} present in the various classes, subclasses and other examples and illustrations set forth elsewhere herein; and wherein Q is OH, Cl, Br, F, I or another good leaving group, e.g., an alkyl or aryl sulfonate, such as a mesylate, tosylate, brosylate, triflate, etc. The following further illustrate such compounds of interest: 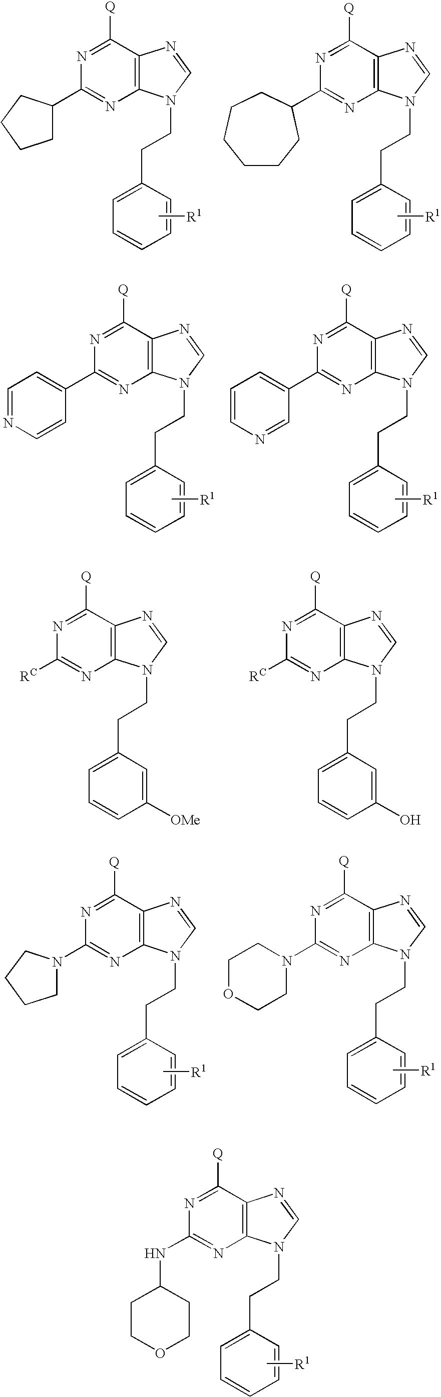
where R¹, R^{C} and Q are as previously defined (e.g., R¹ is H, m-hydroxy, m-methoxy, m-hydroxy-p-fluoro-, etc. as previously described). Preferably Q is halo or hydroxyl. Also of particular interest are the corresponding compounds whose phenethyl aryl ring contains a meta hydroxy (or elaborated derivative thereof, such as an ether, ester, carbamate, etc. as previously discussed) together with an adjacent, ortho-, methyl, ethyl (or other alkyl group) or halo group (again, as previously discussed). One route to substituted phenethylamine intermediates useful for attaching desired R^{A} groups is as follows: 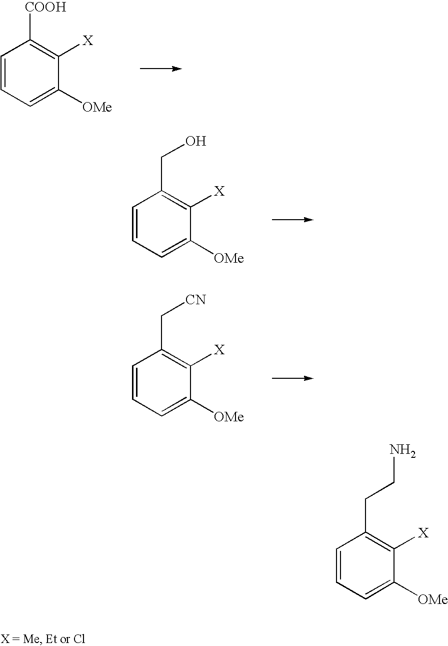
where the appropriate 2-“X”-3-methoxybenzoic acid is reduced to the corresponding alcohol, fitted with a conventional leaving group (e.g. mesylate, bromide, iodide, etc.) and displaced with cyanide to generate the nitrile, which may then be reduced (e.g., with LAH) to yield the phenethylamine. An alternative route starting with aryl aldehydes instead of aryl carboxylic acids is also illustrated in the synthetic examples below.
5. Uses, Formulations, Administration
Pharmaceutical Uses; Indications

This invention provides compounds having biological properties which make them of interest for treating or modulating disease in which kinases may be involved, symptoms of such disease, or the effect of other physiological events mediated by kinases. For instance, a number of compounds of this invention have been shown to inhibit tyrosine kinase activity of Src and abl, among other tyrosine kinases which are believed to mediate the growth, development and/or metastasis of cancer. A number of compounds of the invention have also been found to possess potent in vitro activity against cancer cell lines, including among others K-562 leukemia cells. Observed potencies have been as much as 10-fold more powerful than Gleevec in conventional antiproliferation assays with K562 cells.

Such compounds are thus of interest for the treatment of cancers, including both primary and metastatic cancers, including solid tumors as well as lymphomas and leukemias (including CML, AML and ALL), and including cancers which are resistant to other therapies, including other therapies involving the administration of kinase inhibitors such as Gleevec, Tarceva or Iressa.

Such cancers include, among others, cancers of the breast, cervix, colon and rectum, lung, ovaries, pancreas, prostate, head and neck, gastrointestinal stroma, as well as diseases such as melanoma, multiple myeloma, non-Hodgkin's lymphoma, melanoma, gastric cancers and leukemias (e.g., myeloid, lymphocytic, myelocytic and lymphoblastic leukemias) including cases which are resistant to one or more other therapies, including among others, Gleevec, Tarceva or Iressa.

Resistance to various anticancer agents can arise from one or more mutations in a mediator or effector of the cancer (e.g., mutation in a kinase such as Src or Abl) which correlate with alteration in the protein's drug binding properties, phosphate binding properties, protein binding properties, autoregulation or other characteristics. For example, in the case of BCR-Abl, the kinase associated with chronic myeloid leukemia, resistance to Gleevec has been mapped to a variety of BCR/Abl mutations which are linked to a variety of functional consequences, including among others, steric hindrance of drug occupancy at the kinase's active site, alteration in deformability of the phosphate binding P loop, effects on the conformation of the activation loop surrounding the active site, and others. See e.g. Shah et al, 2002, Cancer Cell 2, 117-125 and Azam et al, 2003, Cell 112, 831-843 and references cited therein for representative examples of such mutations in Bcr/Abl which correlate with drug resistance. See also the following references for additional background information on BCR/Abl, its mechanistic role in CML and drug-resistance-conferring mechanisms and mutations: Kurzrock et al., Philadelphia chromosome-positive leukemias: from basic mechanisms to molecular therapeutics, Ann Intern Med. 2003 May 20;138(10):819-30; O'Dwyer et al., Demonstration of Philadelphia chromosome negative abnormal clones in patients with chronic myelogenous leukemia during major cytogenetic responses induced by imatinib mesylate. Leukemia. 2003 March;17(3):481-7; Hochhaus et al., Molecular and chromosomal mechanisms of resistance to imatinib (STI571) therapy, Leukemia. 2002 November;16(11):2190-6; O'Dwyer et al., The impact of clonal evolution on response to imatinib mesylate (STI571) in accelerated phase CML. Blood. 2002 Sep. 1;100(5):1628-33; Braziel et al., Hematopathologic and cytogenetic findings in imatinib mesylate-treated chronic myelogenous leukemia patients: 14 months' experience. Blood. 2002 Jul. 15;100(2):435-41; Corbin et al., Analysis of the structural basis of specificity of inhibition of the Abl kinase by ST1571. J Biol Chem. 2002 Aug. 30;277(35):32214-9; Wertheim et al., BCR-ABL-induced adhesion defects are tyrosine kinase-independent. Blood. 2002 Jun. 1;99(11):4122-30; Kantarjian et al., Hematologic and cytogenetic responses to imatinib mesylate in chronic myelogenous leukemia, N Engl J Med. 2002 Feb. 28;346(9):645-52. Erratum in: N Engl J Med 2002 Jun. 13;346(24):1923; Hochhaus et al., Roots of clinical resistance to STI-571 cancer therapy. Science. 2001 Sep. 21;293(5538):2163; Druker et al., Activity of a specific inhibitor of the BCR-ABL tyrosine kinase in the blast crisis of chronic myeloid leukemia and acute lymphoblastic leukemia with the Philadelphia chromosome. N Engl J Med. 2001 Apr. 5;344(14):1038-42. Erratum in: N Engl J Med 2001 Jul. 19;345(3):232; Mauro et al., Chronic myelogenous leukemia. Curr Opin Oncol. 2001 January;13(1):3-7. Review; Kolibaba et al., CRKL binding to BCR-ABL and BCR-ABL transformation. Leuk Lymphoma. 1999 March;33(1-2):119-26; Bhat et al., Interactions of p62(dok) with p210(bcr-abl) and Bcr-Abl-associated proteins. J Biol Chem. 1998 Nov. 27;273(48):32360-8; Senechal et al., Structural requirements for function of the Crkl adapter protein in fibroblasts and hematopoietic cells. Mol Cell Biol. 1998 September;18(9):5082-90; Kolibaba et al., Protein tyrosine kinases and cancer. Biochim Biophys Acta. 1997 Dec. 9;1333(3):F217-48. Review; Heaney et al., Direct binding of CRKL to BCR-ABL is not required for BCR-ABL transformation. Blood. 1997 Jan. 1;89(1):297-306; Hallek et al., Interaction of the receptor tyrosine kinase p145c-kit with the p210bcr/abl kinase in myeloid cells. Br J Haematol. 1996 July;94(1):5-16; Oda et al., The SH2 domain of ABL is not required for factor-independent growth induced by BCR-ABL in a murine myeloid cell line. Leukemia. 1995 February;9(2):295-301; Carlesso et al., Use of a temperature-sensitive mutant to define the biological effects of the p210BCR-ABL tyrosine kinase on proliferation of a factor-dependent murine myeloid cell line. Oncogene. 1994 January; 9(1):149-56.

Again, we contemplate that compounds of this invention, both as monotherapies and in combination therapies, will be useful against leukemias and other cancers which are resistant to one or more other anticancer agents, including among others leukemias and other cancers which are resistant in whole or part to other anticancer agents, specifically including Gleevec and other kinase inhibitors, and specifically including leukemias involving one or more mutations in BCR/Abl, within or outside the kinase domain, including but not limited to those noted in any of the foregoing publications. See in particular Azam et al. and references cited therein for examples of such mutations in BCR/Abl, including, among others, mutations in the drug binding cleft, the phosphate binding P loop, the activation loop, the conserved VAVK of the kinase beta-3 sheet, the catalytic alpha-1 helix of the small N lobe, the long alpha-3 helix within the large C lobe, and the region within the C lobe downstream of the activation loop.

Pharmaceutical Methods

The method of the invention comprises administering to a subject in need thereof a therapeutically effective amount of a compound of the invention.

A “therapeutically effective amount” is that amount effective for detectable killing or inhibition of the growth or spread of cancer cells; the size or number of tumors; or other measure of the level, stage, progression or severity of the cancer. The exact amount required will vary from subject to subject, depending on the species, age, and general condition of the subject, the severity of the disease, the particular anticancer agent, its mode of administration, combination treatment with other therapies, and the like.

The compound, or a composition containing the compound, may be administered using any amount and any route of administration effective for killing or inhibiting the growth of tumors or other forms of cancer.

The anticancer compounds of the invention are preferably formulated in dosage unit form for ease of administration and uniformity of dosage. The expression “dosage unit form” as used herein refers to a physically discrete unit of anticancer agent appropriate for the patient to be treated. As is normally the case, the total daily usage of the compounds and compositions of the present invention will be decided by the attending physician using routine reliance upon sound medical judgment. The specific therapeutically effective dose level for any particular patient or organism will depend upon a variety of factors including the disorder being treated; the severity of the disorder; the potency of the specific compound employed; the specific composition employed; the age, body weight, general health, sex and diet of the patient; the route and schedule of administration; the rate of metabolism and/or excretion of the compound; the duration of the treatment; drugs used in combination or coincident with administration of the compound of this invention; and like factors well known in the medical arts.

Furthermore, after formulation with an appropriate pharmaceutically acceptable carrier in a desired dosage, the compositions of this invention can be administered to humans and other animals orally, rectally, parenterally, intracisternally, intravaginally, intraperitoneally, topically (as by transdermal patch, powders, ointments, or drops), sublingually, bucally, as an oral or nasal spray, or the like.

The effective systemic dose of the compound will typically be in the range of 0.01 to 500 mg of compound per kg of patient body weight, preferably 0.1 to 125 mg/kg, and in some cases 1 to 25 mg/kg, administered in single or multiple doses. Generally, the compound may be administered to patients in need of such treatment in a daily dose range of about 50 to about 2000 mg per patient. Administration may be once or multiple times daily, weekly (or at some other multiple-day interval) or on an intermittent schedule. For example, the compound may be administered one or more times per day on a weekly basis (e.g. every Monday) indefinitely or for a period of weeks, e.g. 4-10 weeks. Alternatively, it may be administered daily for a period of days (e.g. 2-10 days) followed by a period of days (e.g. 1-30 days) without administration of the compound, with that cycle repeated indefinitely or for a given number of repititions, e.g. 4-10 cycles. As an example, a compound of the invention may be administered daily for 5 days, then discontinued for 9 days, then administered daily for another 5 day period, then discontinued for 9 days, and so on, repeating the cycle indefinitely, or for a total of 4-10 times.

The amount of compound which will be effective in the treatment or prevention of a particular disorder or condition will depend in part on well known factors affecting drug dosage. In addition, in vitro or in vivo assays may optionally be employed to help identify optimal dosage ranges. A rough guide to effective doses may be extrapolated from dose-response curves derived from in vitro or animal model test systems. The precise dosage level should be determined by the attending physician or other health care provider and will depend upon well known factors, including route of administration, and the age, body weight, sex and general health of the individual; the nature, severity and clinical stage of the disease; the use (or not) of concomitant therapies; and the nature and extent of genetic engineering of cells in the patient.

When administered for the treatment or inhibition of a particular disease state or disorder, the effective dosage of the compound of this invention may vary depending upon the particular compound utilized, the mode of administration, the condition, and severity thereof, of the condition being treated, as well as the various physical factors related to the individual being treated. In many cases, satisfactory results may be obtained when the compound is administered in a daily dosage of from about 0.01 mg/kg-500 mg/kg, preferably between 0.1 and 125 mg/kg, and more preferably between 1 and 25 mg/kg. The projected daily dosages are expected to vary with route of administration. Thus, parenteral dosing will often be at levels of roughly 10% to 20% of oral dosing levels.

When the compound of this invention is used as part of a combination regimen, dosages of each of the components of the combination are administered during a desired treatment period. The components of the combination may administered at the same time; either as a unitary dosage form containing both components, or as separate dosage units; the components of the combination can also be administered at different times during a treatment period, or one may be administered as a pretreatment for the other.

Regarding the Compounds

Compounds of present invention can exist in free form for treatment, or where appropriate, as a pharmaceutically acceptable salt or other derivative. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, phosphonates and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the isolation and purification of the compounds of the invention, or separately by reacting the free base or free acid of a compound of the invention with a suitable base or acid, respectively. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers preferably to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters includes formates, acetates, propionates, butyrates, acrylates and ethylsuccinates. Obviously, esters can be formed with a hydroxyl or carboxylic acid group of the compound of the invention.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are transformed in vivo to yield the parent compound of the above formula, for example by hydrolysis in blood. See, e.g., T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, both of which are incorporated herein by reference.

Compositions

Accordingly, compositions are provided, which comprise any one of the compounds described herein (or a prodrug, pharmaceutically acceptable salt or other pharmaceutically acceptable derivative thereof), and one or more pharmaceutically acceptable carriers or excipients. These compositions optionally further comprise one or more additional therapeutic agents. Alternatively, a compound of this invention may be administered to a patient in need thereof in combination with the administration of one or more other therapeutic regimens (e.g. Gleevec or other kinase inhibitors, interferon, bone marrow transplant, farnesyl transferase inhibitors, bisphosphonates, thalidomide, cancer vaccines, hormonal therapy, antibodies, radiation, etc). For example, additional therapeutic agents for conjoint administration or inclusion in a pharmaceutical composition with a compound of this invention may be another one or more anticancer agents.

As described herein, the compositions of the present invention comprise a compound of the invention together with a pharmaceutically acceptable carrier, which, as used herein, includes any and all solvents, diluents, or other vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition.

Formulations

This invention also encompasses a class of compositions comprising the active compounds of this invention (including those of Formula I, Formula II, Formulas I(a) through I(q) and the other compounds described herein) in association with one or more pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly, intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a tablet, capsule, suspension or liquid. The pharmaceutical composition is preferably made in the form of a dosage unit containing a particular amount of the active ingredient.

Examples of such dosage units are tablets or capsules. For example, these may contain an amount of active ingredient from about 1 to 2000 mg, preferably from about 1 to 500 mg, more commonly from about 5 to 200 mg. A suitable daily dose for a human or other mammal may vary depending on the condition of the patient and other factors, but, once again, can be determined using routine methods.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A typical daily dose is in the range of 0.01 to 500 mg of compound per kg body weight, preferably between 0.1 and 125 mg/kg body weight and in some cases between 1 and 25 mg/kg body weight. As mentioned previously, the daily dose can be given in one administration or may be divided between 2, 3, 4 or more administrations.

For therapeutic purposes, the active compounds of this invention are ordinarily combined with one or more adjuvants, excipients or carriers appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropyl methyl cellulose.

In the case of skin conditions, it may be preferable to apply a topical preparation of compounds of this invention to the affected area two to four times a day.

Formulations suitable for topical administration include liquid or semi-liquid preparations suitable for penetration through the skin (e.g., liniments, lotions, ointments, creams, or pastes) and drops suitable for administration to the eye, ear, or nose. A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

When formulated in an ointment, the active ingredients may be employed with either paraffinic or a water-miscible ointment base. Alternatively, the active ingredients may be formulated in a cream with an oil-in-water cream base. If desired, the aqueous phase of the cream base may include, for example at Least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs.

The compounds of this invention can also be administered by a transdermal device. Preferably transdermal administration will be accomplished using a patch either of the reservoir and porous membrane type or of a solid matrix variety. In either case, the active agent is delivered—continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner.

While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the socalled emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required.

Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients.

The active ingredients are preferably present in such formulations in a concentration of 0.5 to 20%, advantageously 0.5 to 10% and particularly about 1.5% w/w.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers.

Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

For pulmonary administration, the pharmaceutical composition may be administered in the form of an aerosol or with an inhaler including dry powder aerosol.

Suppositories for rectal administration of the drug can be prepared by mixing the drug with a suitable nonirritating excipient such as cocoa butter and polyethylene glycols that are solid at ordinary temperatures but liquid at the rectal temperature and will therefore melt in the rectum and release the drug.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

Pharmaceutical compositions of this invention comprise a compound of the formulas described herein or a pharmaceutically acceptable salt thereof; an additional agent selected from a kinase inhibitory agent (small molecule, polypeptide, antibody, etc.), an immunosuppressant, an anticancer agent, an anti-viral agent, antiinflammatory agent, antifungal agent, antibiotic, or an anti-vascular hyperproliferation compound; and any pharmaceutically acceptable carrier, adjuvant or vehicle.

Alternate compositions of this invention comprise a compound of the formulae described herein or a pharmaceutically acceptable salt thereof; and a pharmaceutically acceptable carrier, adjuvant or vehicle. Such compositions may optionally comprise one or more additional therapeutic agents, including, for example, kinase inhibitory agents (small molecule, polypeptide, antibody, etc.), immunosuppressants, anti-cancer agents, anti-viral agents, antiinflammatory agents, antifungal agents, antibiotics, or anti-vascular hyperproliferation compounds.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, selfemulsifying drug delivery systems (SEDDS) such as d-atocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as u-, P-, and y-cyclodextrin, or chemically modified derivatives such as hydroxyalkylcyclodextrins, including 2 and 3-hydroxypropyl-cyclodextrins, or other solubilized derivatives may also be advantageously used to enhance delivery of compounds of the formulae described herein.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents.

If desired, certain sweetening and/or flavoring and/or coloring agents may be added.

The pharmaceutical compositions of this invention may comprise formulations utilizing liposome or microencapsulation techniques, various examples of which are known in the art.

The pharmaceutical compositions of this invention may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents, examples of which are also well known in the art.

Combinations

While the compounds of the invention can be administered as the sole active pharmaceutical agent, they can also be used in combination with one or more other compounds of the invention or with one or more other agents. When administered as a combination, the therapeutic agents can be formulated as separate compositions that are administered at the same time or sequentially at different times, or the therapeutic agents can be given as a single composition.

The phrase “co-therapy” (or “combination-therapy”), in defining use of a compound of this invention and another pharmaceutical agent, is intended to encompass administration of each agent in a sequential manner in a regimen that will provide beneficial effects of the drug combination, and is intended as well to embrace coadministration of these agents in a substantially simultaneous manner, such as in a single capsule having a fixed ratio of these active agents or in multiple, separate capsules for each agent.

Thus, the administration of compounds of the present invention may be in conjunction with additional therapies known to those skilled in the art in the prevention or treatment of cancer, such as radiation therapy or cytostatic agents, cytotoxic agents, other anti-cancer agents and other drugs to amerliorate symptoms of the cancer or side effects of any of the drugs.

If formulated as a fixed dose, such combination products employ the compounds of this invention within the accepted dosage ranges. Compounds of this invention may also be administered sequentially with other anticancer or cytotoxic agents when a combination formulation is inappropriate. The invention is not limited in the sequence of administration; compounds of this invention may be administered prior to, simulateously with, or after administration of the other anticancer or cytotoxic agent.

Currently, standard treatment of primary tumors consists of surgical excision, when appropriate, followed by either radiation or chemotherapy, typically administered intravenously (IV). The typical chemotherapy regime consists of either DNA alkylating agents, DNA intercalating agents, CDK inhibitors, or microtubule poisons. The chemotherapy doses used are just below the maximal tolerated dose and therefore dose limiting toxicities typically include, nausea, vomiting, diarrhea, hair loss, neutropenia and the like.

There are large numbers of antineoplastic agents available in commercial use, in clinical evaluation and in pre-clinical development, which would be selected for treatment of cancer by combination drug chemotherapy. And there are several major categories of such antineoplastic agents, namely, antibiotic-type agents, alkylating agents, antimetabolite agents, hormonal agents, immunological agents, interferon-type agents and a category of miscellaneous agents.

A first family of antineoplastic agents which may be used in combination with compounds of the present invention includes antimetabolite-type/thymidilate synthase inhibitor antineoplastic agents. Suitable antimetabolite antineoplastic agents may be selected from but not limited to the group consisting of 5-FU-fibrinogen, acanthifolic acid, aminothiadiazole, brequinar sodium, carmofur, CibaGeigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co.

EX-015, fazarabine, floxuridine, fludarabine phosphate, 5fluorouracil, N-(21-furanidyl)fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC788, thioguanine, tiazofurin, Erbamont TIF, trimetrexate, tyrosine kinase inhibitors, Taiho UFT and uricytin.

A second family of antineoplastic agents which may be used in combination with compounds of the present invention consists of alkylating-type antineoplastic agents. Suitable alkylating-type antineoplastic agents may be selected from but not limited to the group consisting of Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D 384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G M, Chinoin GYKI-17230, hepsulfam, ifosfamide, iproplatin, lomustine, mafosfamide, mitolactolf Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

A third family of antineoplastic agents which may be used in combination with compounds of the present invention consists of antibiotic-type antineoplastic agents. Suitable antibiotic-type antineoplastic agents may be selected from but not limited to the group consisting of Taiho 4181-A, aclarubicin, actinomycin D, actinoplanone, Erbamont ADR-456, aeroplysinin derivative, Ajinomoto AN II, Ajinomoto AN3, Nippon Soda anisomycins, anthracycline, azino-mycin-A, bisucaberin, Bristol-Myers BL-6859, Bristol-Myers BMY-25067, Bristol-Myers BNY-25551, Bristol-Myers BNY-26605 IBristolMyers BNY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko, DC89-AI, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-AI, esperamicin-Alb, Erbamont FCE21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

A fourth family of antineoplastic agents which may be used in combination with compounds of the present invention consists of a miscellaneous family of antineoplastic agents, including tubulin interacting agents, topoisomerase II inhibitors, topoisomerase I inhibitors and hormonal agents, selected from but not limited to the group consisting of (xcarotene, (X-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5. antineoplaston AS2-1F Henkel APD, aphidicolin glycinate, asparaginase, Avarol, baccharin, batracylin, benfluron, benzotript, Ipsen-Beaufour BIM-23015, bisantrene, BristoMyers BNY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, WarnerLambert CI-937, Warner-Lambert CI-941, Warner-Lambert CI958, clanfenur, claviridenone, ICN compound 1259, ICN compound 4711, Contracan, Yakult Honsha CPT-11, crisnatol, curaderm, cytochalasin B. cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704t gallium nitrate, genkwadaphnin, Chugai GLA-43, Glaxo GR-63178, grifolan NMF5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU 1121 Lilly LY-186641, NCI (US) MAP, marycin, Merrel Dow MDL-27048, Medco MEDR-340, merbarone, merocyanlne derivatives, methylanilinoacridine, Molecular Genetics MGI136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, WarnerLambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides and Yamanouchi YM Alternatively, the present compounds may also be used in co-therapies with other antineoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon1, interferon alpha, natural, interferon beta, interferon beta-Ia, interferon beta-Ib, interferon gamma, natural interferon gamma-Ia, interferon gamma-Ib, interleukin-I beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama. vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinidel filgrastim SDO1 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM iodine 131 MAb (Techniclone), polymorphic epithelial mucin-yttrium 90 MAb (Antisoma), marimastat, menogaril, mitumomab, motexafin, gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN)y SU 6668 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Treatment Kits

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the substituted purine dosages, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope. Indeed, various modifications of the invention, and many further embodiments thereof, in addition to those shown and described herein, will become apparent to those skilled in the art upon review of this document, including the examples which follow and the references to the scientific and patent literature cited herein. The contents of those cited references are incorporated herein by reference to help illustrate the state of the art. In addition, for purposes of this invention, the chemical elements are identified in accordance with the Periodic Table of the Elements, CAS version, Handbook of Chemistry and Physics, 75^{th} Ed., inside cover. Additionally, general principles of organic chemistry, as well as specific functional moieties and reactivity, are described in “Organic Chemistry”, Thomas Sorrell, University Science Books, Sausalito: 1999, and “Organic Chemistry”, Morrison & Boyd (3d Ed), the entire contents of both of which are incorporated herein by reference.

### EXAMPLES

### Example 1

### Illustrative Synthesis of Substituted Phenethyl Amines

Variously substituted phenethyl amines can be prepared by the following general route: 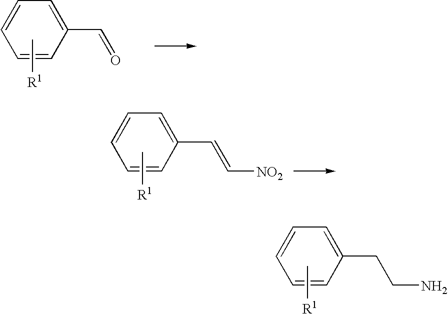
where R¹ represents one or more substituents, protected as desired, as illustrated by the production of the following compounds:

### Example 1(a)

### 2-(4-Fluoro-3-methoxy-phenyl)-ethylamine

### Step 1: 1-Fluoro-2-methoxy-4-(2-nitro-vinyl)-benzene

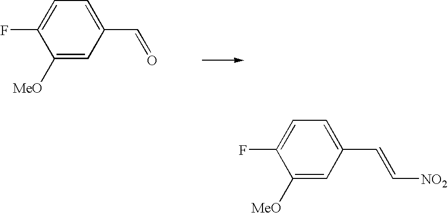

To a mixture of 4-fluoro-3-methoxybenzaldehyde (3.85 g) and nitromethane (1.525 g, in 25 ml of methanol at −10° C. was added a solution of sodium hydroxide (1.15 g in 4 ml) in drops such that the temperature is maintained below 5-10° C. After the addition the mixture was stored at 0° C. This was carefully added to an aqueous hydrochloric acid (conc.HCl 10 ml and water 15 ml). At the end of addition a canary yellow precipitate forms. The mixture was cooled for 30 minutes in ice and then filtered. The filtrate was washed with water and dried in vacuo yielding a yellow solid (3.65 g).

### Step 2: 2-(4-Fluoro-3-methoxy-phenyl)-ethylamine

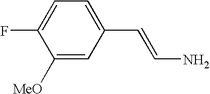

The nitro styrene (3.11 g, 15.77 mmol) prepared as in Step 1 and in 180 ml THF was added dropwise to LAH in THF (63 ml, 63 mmol) cooled in ice. The mixture was then stirred at ice-bath for 15 min and at room temperature for 15 min, and was then refluxed for 2 h. After decomposition of the excess LAH, the amine that was obtained was purified by chromatography on silica gel using ethyl acetate/methanol and triethyl amine, yielding the pure amine pictured above.

### Example 1 (b)

### 2-(4-Chloro-3-methoxy-phenyl)-ethylamine

The title compound is prepared by the method of Example 1 (a), substituting 4-chloro-3-methoxybenzaldehyde for 4-fluoro-3-methoxybenzaldehyde.

### Example 1(c)

### 2-(4-Fluoro-3-methoxy-5-methyl-phenyl)-ethylamine

The title compound is prepared by the method of Example 1 (a), substituting 4-cfluoro-3-methoxy-5-methyl-benzaldehyde for 4-fluoro-3-methoxybenzaldehyde.

Alternative Route: As discussed previously, one may also use the routine transformations of the following route to make variously substituted phenethylamines, particularly in cases in which the appropriately substituted benzoic acid is commercially available or otherwise conveniently obtainable:

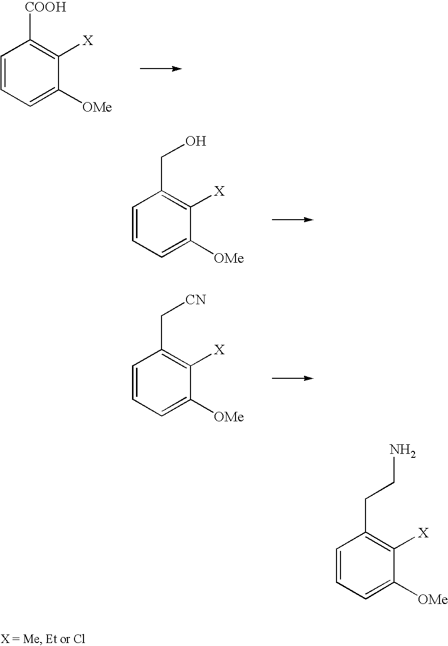

Illustrative mono-, di- and tri-substituted phenethyl amines which may be produced by analogous means to the various methods noted above, are shown below:

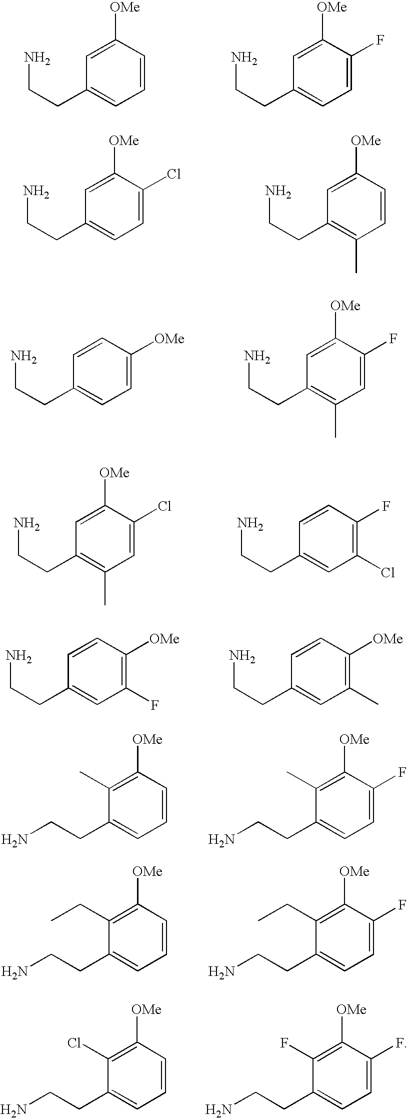

### Example 2

### Preparation of Substituted N-phenethyl Imidazoles

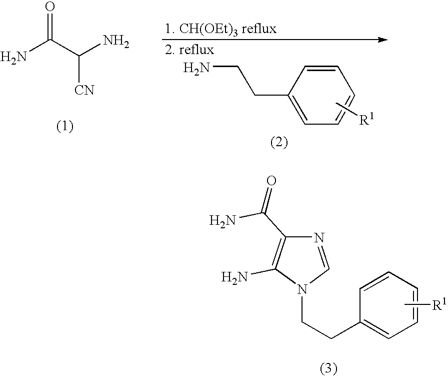

2-amino-2-cyanoacetoamide (40 g) is dissolved in acetonitrile (600 ml) in a 2 L flask and refluxed with triethyl orthoformate (74 ml, 1.1 equivalent) (under N₂) for 75 min. The color of the mixture changes from light brown to pinkish red. This solution is allowed to cool, then the phenethylamine (1.1 equivalent), substituted as desired and with any appropriate protecting groups, is added. An additional 20 ml of acetonitrile is used to complete the transfer of the amine. This mixture is brought back to reflux. After 15 min, precipitate starts to form, and by 30 min all or substantially all starting material is consumed. Reflux may be continued for an additional 30 minutes.

Heating is then switched off. Nitrogen is blown into the flask, still sitting in the heating mantle. After 45 min, the mixture is transferred to a beaker and cooled in ice-bath for 1 h., and the product is recovered. In the case of a solid product, when the total volume is reduced to ˜640 ml, the mixture is filtered through a Buchner funnel, and the filtrate is washed with 200 ml×2 cold acetonitrile and dried. Product may thus be obtained in yields in the mid 60% range.

| The following compounds | |
|---|---|
| may thus be prepared | from these phenethylamines |
| 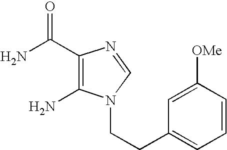 | 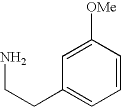 |
| 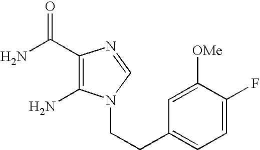 | 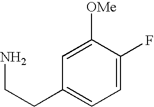 |
| 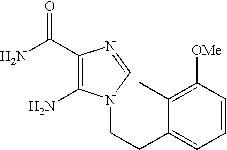 | 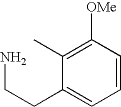 |
| 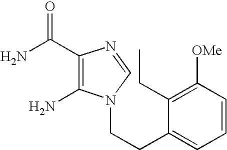 | 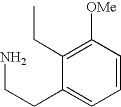 |
| 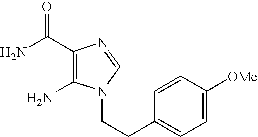 | 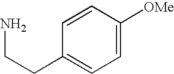 |
| 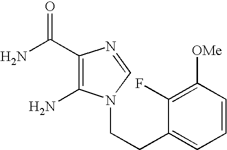 | 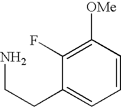 |
| 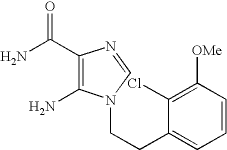 | 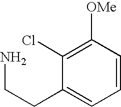 |
| 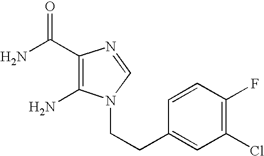 | 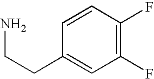 |

### Example 3

### Preparation of Substituted 6-hydroxy Purines from Substituted Phenethyl Imidazoles

As illustrated below, the desired hydroxypurines may be prepared from the corresponding phenethyl imidazole and acyl chloride as follows:

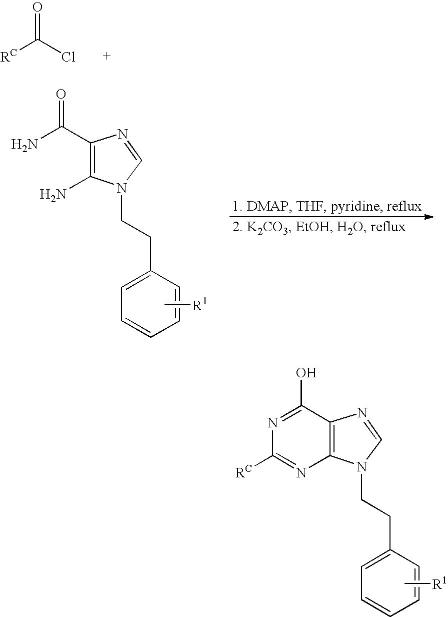

### Example 3(a)

Thus, where R^{C} is cyclopentyl and R¹ is m-methoxy:

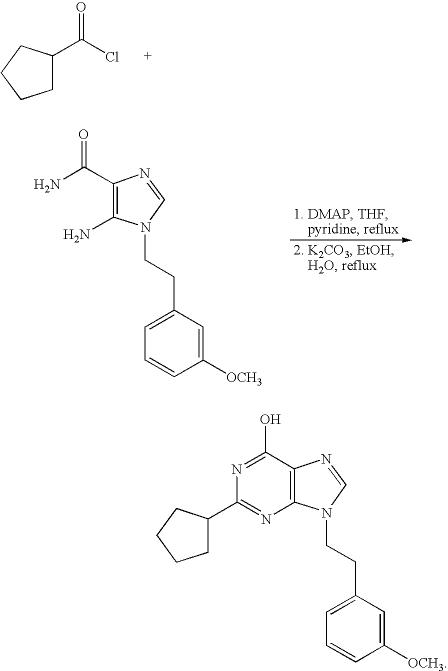

DMAP (0.81 g, 0.05 equiv), THF (300 ml) and Pyridine (150 ml) were added to imidazole (34.6 g, 1.0 equiv) in a 3 L flask, and the mixture was cooled in ice. Cyclopentylcarbonyl chloride (32.3 ml, 2.0 equiv) was added to the mixture dropwise, and the reaction mixture was stirred at that temperature for 15 min after the addition. The reaction mixture was then refluxed for 4 h. Pyridine and THF were removed in vacuo. The residue was dissolved in ethanol/water (2.1 liters, 50:50 EtoH:water) and potassium carbonate (92 g, 5 equiv) was added to this mixture which was then refluxed for 18 h. The reaction mixture was concentrated until most of the ethanol was removed and was then cooled and filtered. The residue was washed with 2×100 mL saturated sodium bicarbonate solution, followed by potassium hydrogen sulfate 2×100 mL and then by water 2×100 mL and dried. Grayish white shining powder, homogeneous by HPLC, was obtained.

### Example 3(b)

The following compound: 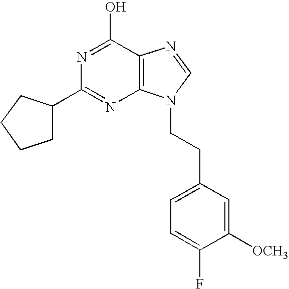
may be obtained by the method of Example 3(a), substituting the 4-fluoro-3-methoxy-phenethyl-substituted imidazole for the 3-methoxy-phenethyl-substituted imidazole.

### Example 3(c)

The following compound: 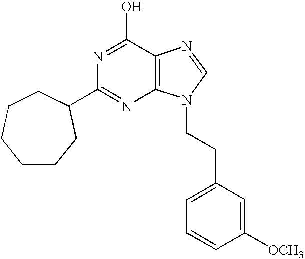
may be obtained by the method of Example 3(a), substituting cycloheptylcarbonyl chloride for cyclopentylcarbonyl chloride.

### Example 3(d)

The following compound: 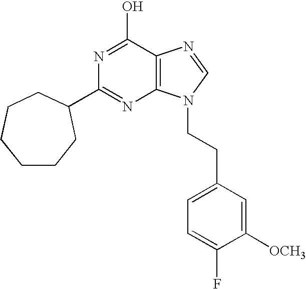
may be obtained by the method of Example 3(a), substituting cycloheptylcarbonyl chloride for cyclopentylcarbonyl chloride.

### Example 3(e)

The following compounds: 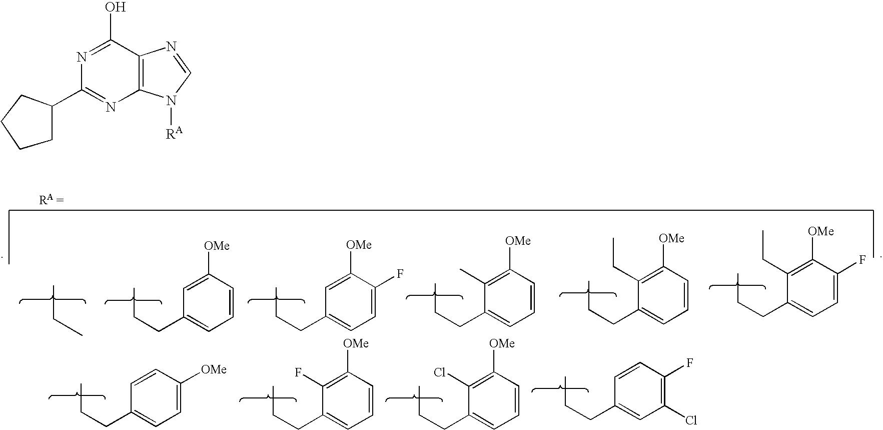
may be obtained by the method of Example 3(a), substituting ethyl imidazole or the appropriately substituted phenethyl imidazole for the 3-methoxy-phenethyl-substituted imidazole.

### Example 4

### Conversion of 6-hydroxypurines to 6-chloropurines

6-hydroxypurines, such as produced in Example 3 above, may be converted to the corresponding 6-chloropurines by the following route:

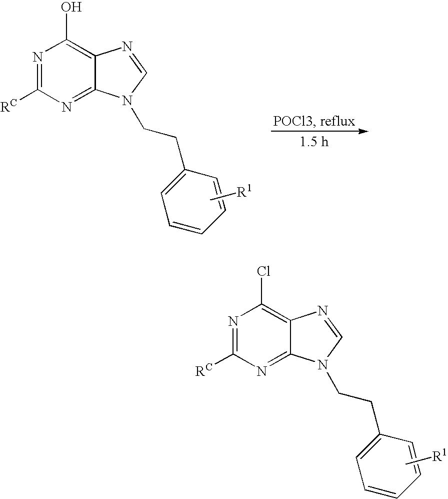

### Example 4(a)

For example, the following transformation is conducted as described below:

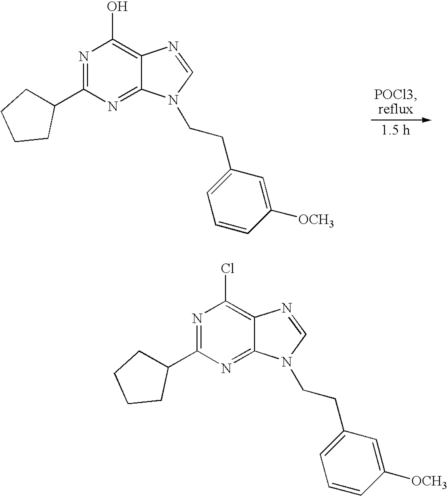

To the 6-hydroxypurine compound (1.5 g), prepared as in Example 3(a), was added phosphorus oxychloride (1.5 mL) under nitrogen and the mixture was refluxed for 1.5 h. Excess phosphorus oxychloride was distilled off in vacuo. To the residue was added a solution of sodium bicarbonate (10%, 50 mL), and the resultant mixture was stirred at room temperature for 30 min. The suspension was extracted with dichloromethane (30 mL×3). The combined dichloromethane extract was dried (Na₂SO₄) and concentrated. The residue was purified by column chromatography to give the depicted 6-chloropurine compound (1.25 g) as a pale yellow foam.

### Example 5

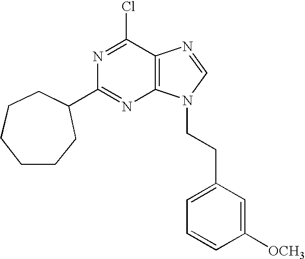

The cycloheptyl analog is produced by the method of Example 4(a), but starting with the corresponding cycloheptyl 6-hydroxypurine in place of the cyclopentyl analog.

Other compounds which may be analogously produced include, among others, the following:

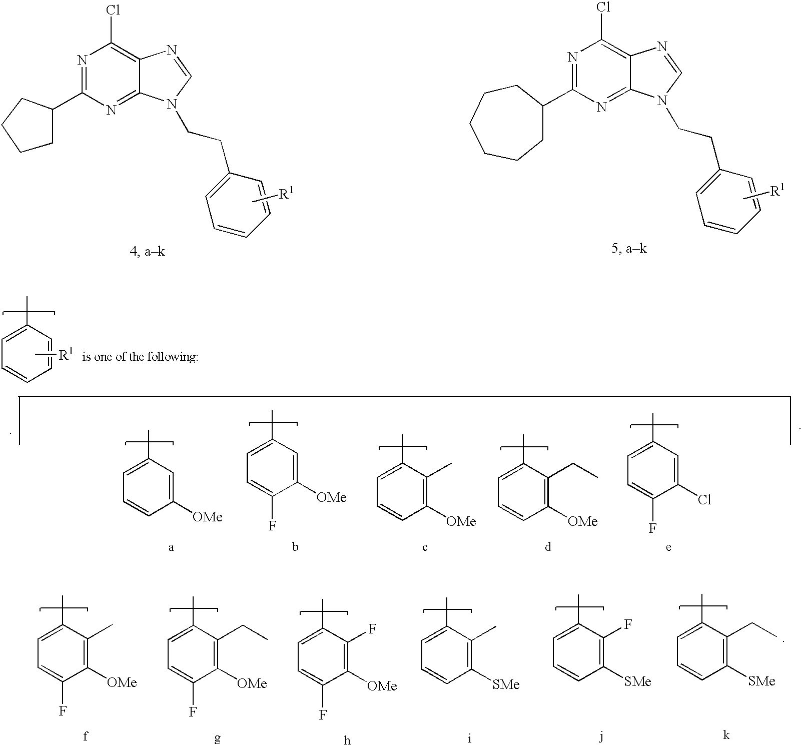

### Example 6

### (4-Aminophenyl)phosphine oxides may be Prepared by the Following Synthetic Scheme

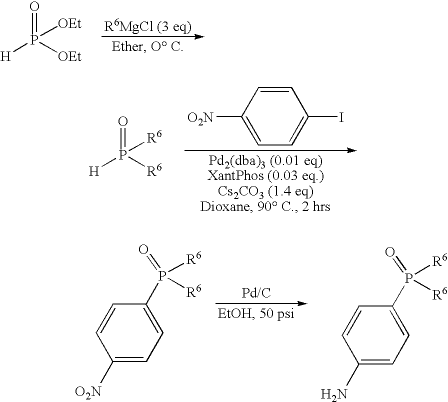

### Example 6(a)

### Synthesis of dimethyl(4-aminophenyl)phosphine oxide

### Synthesis of dimethylphosphine oxide

Methylmagnesium chloride (800 ml, 2.4 mol, 3.0 M in THF) was transferred to a 3 L 3-neck round bottom flask equipped with a mechanic stirrer and a digital thermometer probe. The flask was flushed with nitrogen cooled in an ice/water bath. Diethyl phosphite (110.5 g, 0.8 mol) was added to the solution dropwise during 2 hours. After stirring at room temperature for 30 min, a potassium carbonate solution (331.2 g in 400 ml water) was added slowly to the solution. White solid appears and then ethanol (800 ml) was added. The white suspension was filtered and washed with ethanol (800 ml×2). The filtrate was concentrated under reduced pressure and the residue was added toluene (250 ml) and was concentrated again. The evaporation was repeated 6 more times until constant weight. The product is a thick liquid (55.4 g), used in the next step without further purification.

### Synthesis of dimethyl(4-nitrophenyl)phosphine-oxide

1-Iodo-4-nitrobenzene (1.52 g, 6.1 mmol), dimethylphosphine oxide (500 mg, 6.4 mmol, 1.05 eq), XantPhos (106 mg, 0.183 mmol, 0.03 eq), Tris(dibenzylideneacetone)dipalladium (0) (Pd₂(dba)₃) (56 mg, 0.061 mmol, 0.01 eq) and cesium carbonate (2.78 g, 8.54 mmol, 1.4 eq) was dissolved in 10 ml dry dioxane in a pressure tube under nitrogen. The reaction was heated to 90° C. and stirring was continued at that temperature for 2 hrs. After cooling to room temperature, the reaction mixture was diluted with ethyl acetate (20 ml) and filtered through celite, washed with ethyl acetate (10 ml). The filtrate was evaporated on a rotavapor and the residue was purified by column chromatography on silica gel (10˜15% MeOH in ethyl acetate). After the product fractions were collected and evaporated, the product was obtained as a greenish yellow solid (1.02 g).

### Synthesis of dimethyl(4-aminophenyl)phosphine oxide

Dimethyl(4-nitrophenyl)phosphine-oxide (5.7 g, 28.6 mmol) was dissolved in ethanol (20 ml) in a hydrogenation flask and was added 600 mg of Palladium (10% on activated carbon). The mixture was hydrogenated at room temperature overnight (50 psi). The mixture was filtered through celite and filtrate was evaporated to give a dark residue which was purified by flash column chromatography on silica gel (5% to 10% MeOH/methylene chloride). The product was obtained as a light brown solid (4.8 g).

### Example 6(b)

### Synthesis of dipropyl(4-aminophenyl)phosphine oxide

### Synthesis of dipropylphosphine oxide

Propylmagnesium chloride (150 ml, 0.3 mol, 2.0 M in ether) was transferred to a 500 ml round bottom flask under nitrogen, and the flask was cooled in an ice/water bath. Diethyl phosphite (13.8 g, 0.1 mol) was added to the solution dropwise during 45 minutes. After stirring at room temperature for 30 min, a potassium carbonate solution (41.4 g in 50 ml water) was added slowly to the solution. White solid appears and then ethanol (100 ml) was added. The white suspension was filtered and washed with ethanol (50 ml). The filtrate was concentrated under reduced pressure and to the residue was added chloroform (200 ml) and brine (30 ml). The organic layer separated and dried (MgSO₄). After filtration and evaporation, a white solid obtained (13.0 g). The crude product can be further purified by recrystalization from petroleum ether or hexanes.

### Synthesis of dipropyl(4-nitrophenyl)phosphine-oxide

1-Iodo-4-nitrobenzene (39.7 g, 0.16 mol), dipropylphosphine oxide (22.47 g, 0.168 mol, 1.05 eq), XantPhos (2.77 g, 4.8 mmol, 0.03 eq), Tris(dibenzylideneacetone)dipalladium (0) (Pd₂(dba)₃) (1.46 g, 1.6 mmol, 0.01 eq) and cesium carbonate (72.6 g, 0.22 mol, 1.4 eq) was dissolved in 500 ml dry dioxane in 1 L round bottom flask under nitrogen. The reaction was heated to 90° C. and stirring was continued at that temperature for 2 hrs. After cooling to room temperature, the reaction mixture was diluted with ethyl acetate (300 ml) and filtered through celite, washed with ethyl acetate (200 ml). The filtrate was evaporated on a rotavapor and the residue was purified by column chromatography on silica gel (5% MeOH in methylene chloride). After the product fractions were collected and evaporated, the product was obtained as a brown solid (39 g).

### Synthesis of dipropyl(4-aminophenyl)phosphine oxide

Dipropyl(4-nitrophenyl)phosphine-oxide (26.8 g) was dissolved in ethanol (500 ml) in a 1 L hydrogenation flask and was added 2.7 g of Palladium (10% on activated carbon). The mixture was hydrogenated at room temperature overnight (50 psi). The mixture was filtered through celite and filtrate was evaporated to give a dark residue which was purified by flash column chromatography on silica gel (5% to 10% MeOH/methylene chloride). The product was obtained as an orange solid (20.5 g).

### Example 6(c)

### Synthesis of dialkylyl(4-aminopyridylyl)phosphine oxides

Corresponding pyridyl compounds may be prepared as described above, but starting with halo, nitro pyridine instead of halo, nitro benzene:

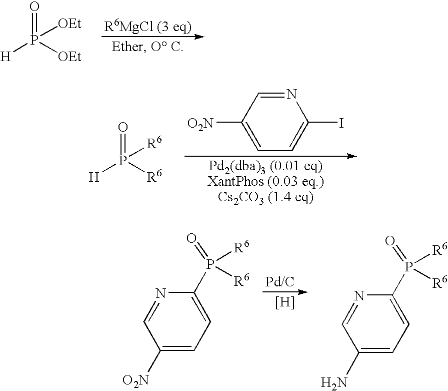

### Example 7

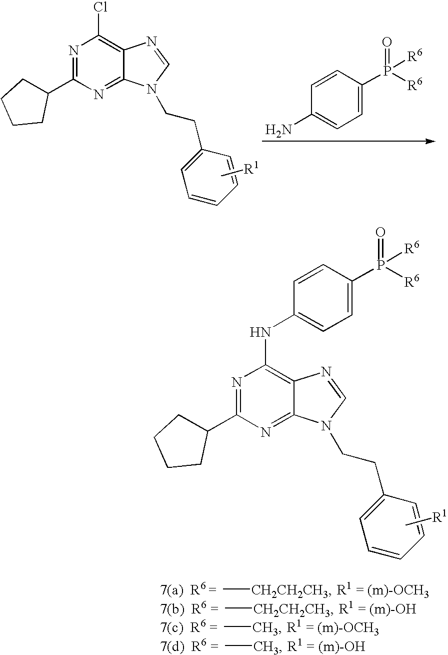

### Example 7(a)

### 2-cyclopentanyl-6-N-(4-phophinodipropyl)phenyl-9-_-(4-fluoro-3-methoxyphenethyl)purine

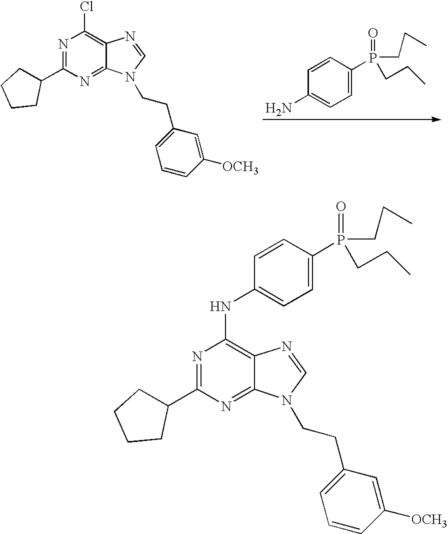

To the 6-chloro purine derivative (1.01 mmol), 4-phosphono-dipropyl aniline (1 mmol), Tris(dibenzylideneacetone)dipalladium (0) (0.015 mmol) 2-(Dicyclohexylphosphino)biphenyl (0.04 mmol) in acetonitrile (10 ml) was added potassium phosphate (1.2 mmol) and the whole reaction mixture was flushed with argon for 10 minute and sealed and heated at 90° C. for 18 h when the HPLC of the reaction mixture shows no starting materials. Reaction mixture was filtered over celite and the celite was repeatedly washed with dichloromethane. Combined organic phase was concentrated and purified by flash chromatography using ethyl acetate/methanol (10%) to give the 6-amino purine derivative in 70% yield.

### Example 7(b)

### 2-cyclopentanyl-6-N-(4-phophinodipropyl)phenyl-9-_-(4-fluoro-3-hydoxyphenethyl purine

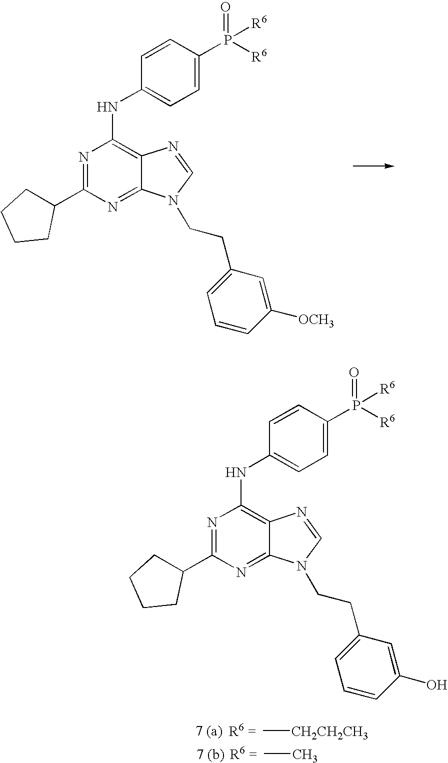

To 2.92 mmol of the methoxy compound produced in Example 7(a) in DCM (50 ml) at 0C was added a DCM solution of BBr3 (5.87 ml, 5.87 mmol) and it was allowed to come to rt and stirred at this temperature for 18 h. Reaction was quenched with 10% sodium carbonate and reextracted with ethyl acetate (3×30 ml). Ethyl acetate was concentrated. The residue was treated with TFA (10 ml) and Conc. HCl (10 ml) and stirred at rt over night to decompose the boronate. After evaporating the acids the residue was neutralized with 10% sodium carbonate and extracted with ethyl acetate (3×30 ml). After drying the ethyl acetate over sodium sulfate, and concentrating, the residue was purified by flash chromatography using ethyl acetate/methanol (10%) gave the phenolic derivative as a nice off-white solid.

### Example 8

### Carbamates

Carbamates of hydroxyphenethyl purines may be prepared by conversion of the hydroxyl group to an activated carbonate, followed by reaction with the appropriate amine: 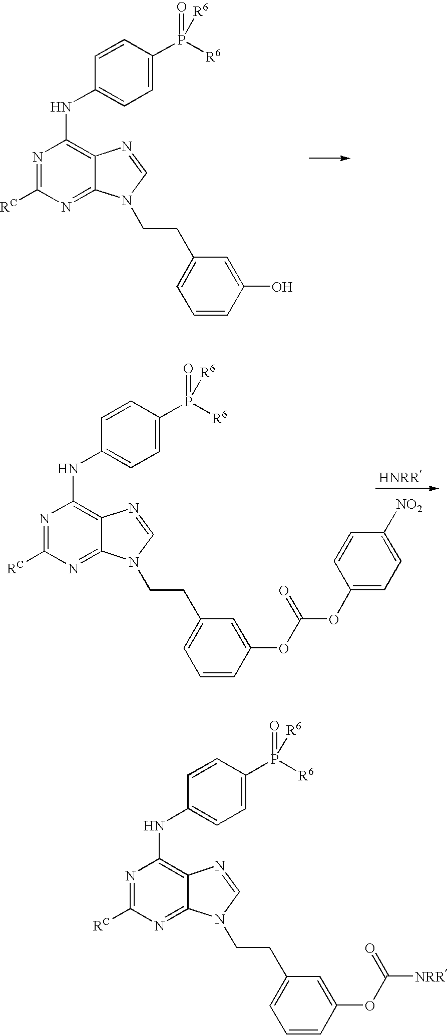
as illustrated below:

### 3-(2-{(2-Cyclopentyl-6-[4-(dimethyl-phosphinoyl)-phenylamino]-purin-9-yl}-ethyl)-phenoxy-N,N-ethylmethyl carbamate (c)

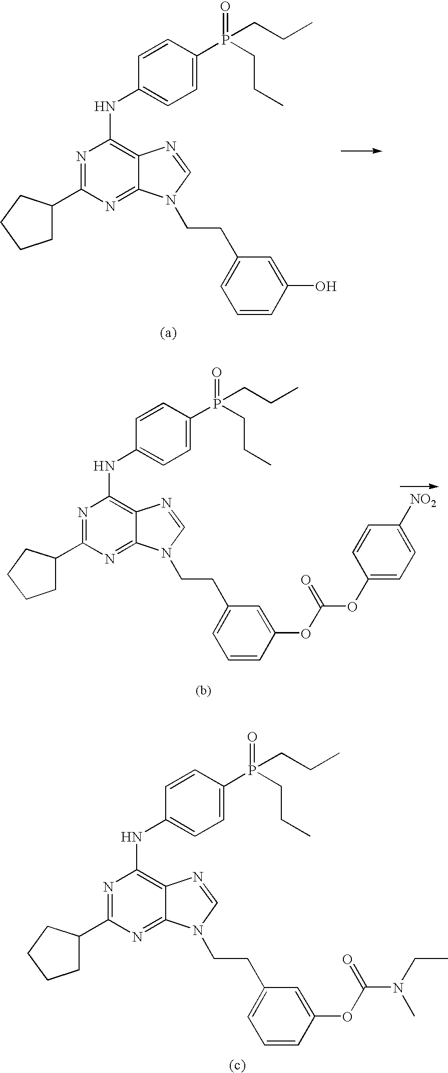

To a cooled (0° C.) stirring solution of 6.0 g (0.011 mol) of 3-(2-{2-Cyclopentyl-6-[4-(dipropyl-phosphinoyl)-phenylamino]-purin-9-yl}-ethyl)-phenol (a) and triethylamine (3.5 mL, 0.025 mol) in 110 mL of DCM was added 2.5 g (0.012 mol) of solid 4-nitrophenyl chloroformate portionwise over ˜5 min under a N₂ atmosphere. The resulting yellow solution was stirred at 0° C. under an atmosphere of N₂ for ˜5 min, the ice bath was removed and stirring was continued at ambient temperature for 2 h. The reaction solution was partitioned between DCM (500 mL) and pH=2-3 dilute aq HCl (200 mL), upon which the layers were separated. The organic layer was repeatedly washed with pH=2-3 dilute aq HCl (5×200 mL), the combined organics were washed with brine (2×200 mL) then dried over MgSO₄ and concentrated. The crude product was purified by silica gel flash chromatography (eluted with 2% iPrOH/DCM then 5% iPrOH/DCM) to provide 4.75 g of a white (slightly yellow) solid: ³¹P NMR (121 MHz, DMSO-d₆)_{—}43.387. 697 m/z (M+H).

To a stirred solution of 0.2 g (0.29 mmol) of 3-(2-{2-Cyclopentyl-6-[4-(dimethyl-phosphinoyl)-phenylamino]-purin-9-yl}-ethyl)-phenoxy-p-nitrophenyl carbonate (b) in 2.5 mL of DCM, under an atmosphere of N₂, was added dropwise a solution of N-ethylmethylamine (0.085 g, 1.43 mmol) in 0.5 mL of DCM. The resulting yellow solution was stirred at ambient temperature for 3 h. The reaction solution was partitioned between DCM (150 mL) and pH=2-3 dilute aq HCl (50 mL), upon which the layers were separated. The organic layer was repeatedly washed with pH=2-3 dilute aq HCl (2×50 mL), the combined organics were washed with brine (50 mL) then dried over MgSO₄ and concentrated. The crude product (c) was purified by silica gel flash chromatography (eluted with 5% iPrOH/DCM then 10% iPrOH/DCM) to provide 0.112 g of a white solid: ³¹P NMR (121 MHz, DMSO-d₆)_{—}43.390. 617 m/z (M+H).

The following analogs of Compound (c) were made by analogous means using the appropriate amine in place of the N-methyl, N-ethyl carbamate of Compound (c):

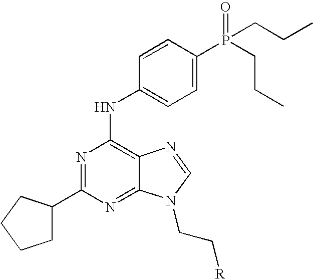

| Compound | R |
|---|---|
| d | 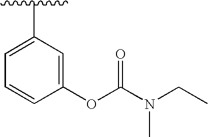 |
| e | 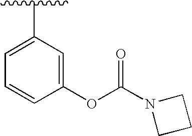 |
| f | 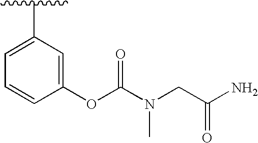 |
| g | 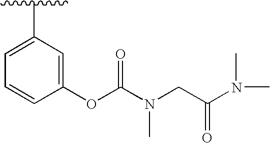 |
| h | 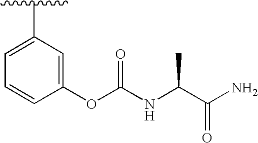 |
| i | 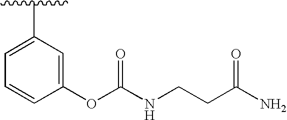 |
| j | 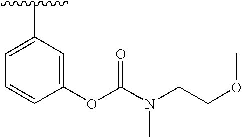 |
| k | 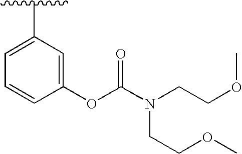 |
| l | 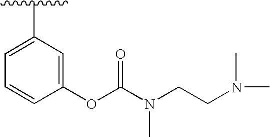 |
| m | 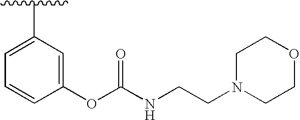 |
| n | 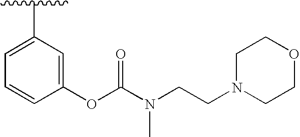 |

The following may be prepared by analgous means:

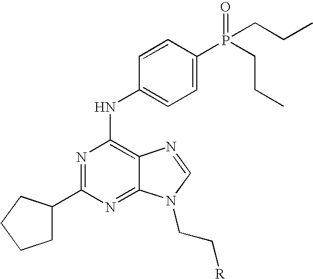

| Compound | R |
|---|---|
| o | 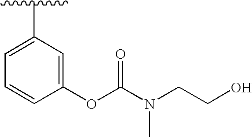 |
| p | 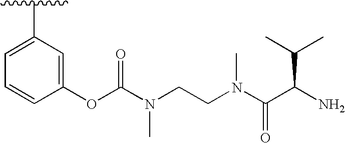 |
| q | 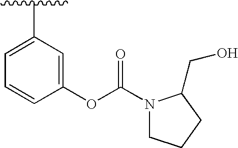 |
| r | 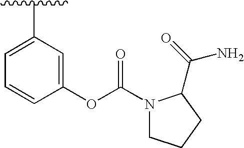 |
| s | 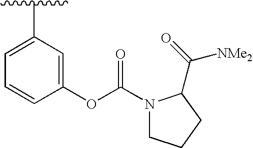 |
| t | 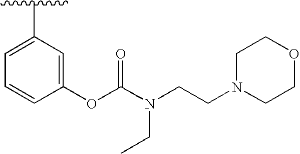 |
| u | 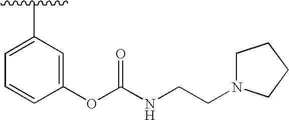 |
| v | 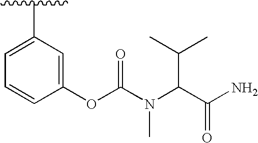 |
| w | 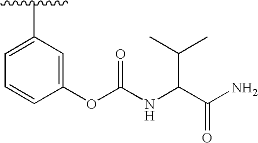 |
| x | 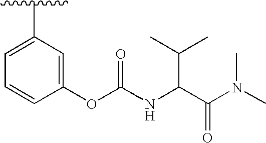 |
| y | 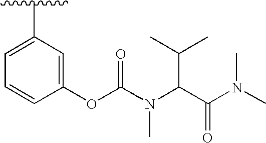 |

### Example 9

### Biological Evaluation of Compounds

Compounds of this invention may be evaluated in a variety of assays to determine their biological activities. For example, the compounds of the invention can be tested for their ability to inhibit various protein kinases of interest. The compounds can also be evaluated for their cytotoxic and growth inhibitory effects on tumor cells of interest. See e.g., WO 03/000188, pages 115-136, the full contents of which are incorporated herein by reference.

Kinase Inhibition

More specifically, the compounds described herein are screened for kinase inhibition activity as follows. Kinases suitable for use in the following protocol include, but are not limited to: Abl, Lck, Lyn, Src, Fyn, Syk, Zap-70, Itk, Tec, Btk, EGFR, ErbB2, Kdr, Flt1, Flt-3, Tek, c-Met, InsR, and AKT.

Kinases are expressed as either kinase domains or full length constructs fused to glutathione S-transferase (GST) or polyHistidine tagged fusion proteins in either E. coli or Baculovirus-High Five expression systems. They are purified to near homogeneity by affinity chromatography as previously described (Lehr et al., 1996; Gish et al., 1995). In some instances, kinases are co-expressed or mixed with purified or partially purified regulatory polypeptides prior to measurement of activity.

Kinase activity and inhibition are measured by established protocols (see e.g., Braunwalder et al., 1996). Briefly, the transfer of ³³PO₄ from ATP to the synthetic substrates poly(Glu, Tyr) 4:1 or poly(Arg, Ser) 3:1 attached to the bioactive surface of microtiter plates is taken as a measure of enzyme activity. After an incubation period, the amount of phosphate transferred is measured by first washing the plate with 0.5% phosphoric acid, adding liquid scintillant, and then counting in a liquid scintillation detector. The IC50 is determined by the concentration of compound that causes a 50% reduction in the amount of ³³P incorporated onto the substrate bound to the plate.

Other methods relying upon the transfer of phosphate to peptide or polypeptide substrate containing tyrosine, serine, threonine or histidine, alone, in combination with each other, or in combination with other amino acids, in solution or immobilized (i.e., solid phase) are also useful.

For example, transfer of phosphate to a peptide or polypeptide can also be detected using scintillation proximity, Fluorescence Polarization and homogeneous time-resolved fluorescence. Alternatively, kinase activity can be measured using antibody-based methods in which an antibody or polypeptide is used as a reagent to detect phosphorylated target polypeptide.

For additional background information on such assay methodologies, see e.g., Braunwalder et al., 1996, Anal. Biochem. 234(I):23; Cleaveland et al., 1990, Anal Biochem. 190(2):249 Gish et al. (1995). Protein Eng. 8(6):609 Kolb et al. (1998). Drug Discov. Toda V. 3:333 Lehr et al. (1996). Gene 169(2):27527-87 Seethala et al. (1998). Anal Biochem. 255(2):257 Wu et al. (2000).

IC50 values in the single digit nanomolar and subnanomolar (i.e., less than 1 nM) range have been observed for compounds of this invention against Src kinase.

Cell-Based Assays

Certain compounds of this invention have also been demonstrated cytotoxic or growth inhibitory effects on tumor and other cancer cell lines and thus may be useful in the treatment of cancer and other cell proliferative diseases. Compounds are assayed for anti-tumor activity using in vivo and in vitro assays which are well known to those skilled in the art. Generally, initial screens of compounds to identify candidate anti-cancer drugs are performed in cellular assays. Compounds identified as having anti-proliferative activity in such cell-based assays can then be subsequently assayed in whole organisms for anti-tumor activity and toxicity. Generally speaking, cell-based screens can be performed more rapidly and cost-effectively relative to assays that use whole organisms. For purposes of this invention, the terms “anti-tumor” and “anti-cancer” activity are used interchangeably.

Cell-based methods for measuring antiproliferative activity are well known and can be used for comparative characterization of compounds of this invention. In general, cell proliferation and cell viability assays are designed to provide a detectable signal when cells are metabolically active. Compounds may be tested for antiproliferative activity by measuring any observed decrease in metabolic activity of the cells after exposure of the cells to compound. Commonly used methods include, for example, measurement of membrane integrity (as a measure of cell viability)(e.g. using trypan blue exclusion) or measurement of DNA synthesis (e.g. by measuring incorporation of BrdU or 3H-thymidine).

Some methods for assaying cell proliferation use a reagent that is converted into a detectable compound during cell proliferation. Particularly preferred compounds are tetrazolium salts and include without limitation MTT (3-(4,5-dimethylthiazol-2-yl)-2,5-diphenyltetrazolium bromide; Sigma-Aldrich, St. Louis, Mo.), MTS (3-(4,5-dimethylthiazol-2-yl)-5-(3-carboxymethoxyphenyl)-2-(4-sulfophenyl)-2H-tetrazolium), XTT (2,3-bis(2-Methoxy-4-nitro-5-sulfophenyl)-2H-tetrazolium-5-carboxanilide), INT, NBT, and NTV (Bernas et al. Biochim Biophys Acta 1451(1):73-81, 1999). Preferred assays utilizing tetrazolium salts detect cell proliferation by detecting the product of the enzymatic conversion of the tetrazolium salts into blue formazan derivatives, which are readily detected by spectroscopic methods (Mosman. J. Immunol. Methods. 65:55-63,1983).

Generally, preferred methods for assaying cell proliferation involve incubating cells in a desired growth medium with and without the compounds to be tested. Growth conditions for various prokaryotic and eukaryotic cells are well-known to those of ordinary skill in the art (Ausubel et al. Current Protocols in Molecular Biology. Wiley and Sons. 1999; Bonifacino et al. Current Protocols in Cell Biology. Wiley and Sons. 1999 both incorporated herein by reference). To detect cell proliferation, the tetrazolium salts are added to the incubated cultured cells to allow enzymatic conversion to the detectable product by active cells. Cells are processed, and the optical density of the cells is determined to measure the amount of formazan derivatives. Furthermore, commercially available kits, including reagents and protocols, are availabe for examples, from Promega Corporation (Madison, Wis.), Sigma-Aldrich (St. Louis, Mo.), and Trevigen (Gaithersburg, Md.).

A wide variety of cell types may be used to screen compounds for antiproliferative activity, including the following cell lines, among others: COLO 205 (colon cancer), DLD-1 (colon cancer), HCT-15 (colon cancer), HT29 (colon cancer), HEP G2 (Hepatoma), K-562 (Leukemia), A549 (Lung), NCI-H249 (Lung), MCF7 (Mammary), MDA-MB-231 (Mammary), SAOS-2 (Osteosarcoma), OVCAR-3 (Ovarian), PANC-1 (Pancreas), DU-145 (Prostate), PC-3 (Prostate), ACHN (Renal), CAKI-1 (Renal), MG-63 (Sarcoma).

Preferably, the cell line is a mammalian, but is not limited to mammalian cells since lower order eukaryotic cells such as yeast may also be used to screen compounds. Preferred mammalian cell lines are derived from humans, rats, mice, rabbits, monkeys, hamsters, and guinea pigs since cells lines from these organisms are well-studied and characterized. However, others may be used as well.

Suitable mammalian cell lines are often derived from tumors. For example, the following tumor cell-types may be sources of cells for culturing cells: melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Non-limiting examples of mammalian cells lines that have been widely used by researchers include HeLa, NIH/3T3, HT1080, CHO, COS-1, 293T, WI-38 and CV1/EBNA-1.

Other cellular assays may be used which rely upon a reporter gene to detect metabolically active cells. Non-limiting examples of reporter gene expression systems include green fluorescent protein (GFP), and luciferase. As an example of the use of GFP to screen for potential antitumor drugs, Sandman et al. (Chem Biol. 6:541-51; incorporated herein by reference) used HeLa cells containing an inducible variant of GFP to detect compounds that inhibited expression of the GFP, and thus inhibited cell proliferation.

Compounds identified by such cellular assays as having anti-cell proliferation activity are then tested for anti-tumor activity in whole organisms. Preferably, the organisms are mammalian. Well-characterized mammalians systems for studying cancer include rodents such as rats and mice. Typically, a tumor of interest is transplanted into a mouse having a reduced ability to mount an immune response to the tumor to reduce the likelihood of rejection. Such mice include for example, nude mice (athymic) and SCID (severe combined immunodeficiency) mice. Other transgenic mice such as oncogene containing mice may be used in the present assays (see for example U.S. Pat. No. 4,736,866 and U.S. Pat. No. 5,175,383). For a review and discussion on the use of rodent models for antitumor drug testing see Kerbel (Cancer Metastasis Rev. 17:301-304,1998-99).

In general, the tumors of interest are implanted in a test organism preferably subcutaneously. The organism containing the tumor is treated with doses of candidate anti-tumor compounds. The size of the tumor is periodically measured to determine the effects of the test compound on the tumor. Some tumor types are implanted at sites other than subcutaneous sites (e.g. intraperitoneal sites) and survival is measured as the endpoint. Parameters to be assayed with routine screening include different tumor models, various tumor and drug routes, and dose amounts and schedule. For a review of the use of mice in detecting antitumor compounds see Corbett et al. (Invest New Drugs. 15:207-218, 1997; incorporated herein by reference).

Compounds of this invention which were tested for antiproliferative activity against the human K562 leukemia cell line gave IC50 values of ½ to ˜1/10 that of Gleevec, which was used as the control. In other words, the compounds tested were 2 to about 10 times more potent than Gleevec in this assay.

### Example 10

### Pharmaceutical Compositions

Representative pharmaceutical dosage forms of the compounds of this invention (the active ingredient being referred to as “Compound”), for therapeutic or prophylactic use in humans:

| (a) Tablet I | mg/tablet | |
|---|---|---|
| Compound | 100 | |
| Lactose Ph.Eur | 182.75 | |
| Croscarmellose sodium | 12.0 | |
| Maize starch paste (5% w/v paste) | 2.25 | |
| Magnesium stearate | 3.0 | |
| (b) Tablet II | mg/tablet | |
| Compound | 50 | |
| Lactose Ph.Eur | 223.75 | |
| Croscarmellose sodium | 6.0 | |
| Maize starch | 15.0 | |
| Polyvinylpyffolidone (5% w/v paste) | 2.25 | |
| Magnesium stearate | 3.0 | |
| (c) Tablet III | mg/tablet | |
| Compound | 1.0 | |
| Lactose Ph.Eur | 93.25 | |
| Croscarmellose sodium | 4.0 | |
| Maize starch paste (5% w/v paste) | 0.75 | |
| Magnesium stearate | 1.0-76 | |
| (d) Capsule | mg/capsule | |
| Compound | 10 | |
| Lactose Ph.Eur | 488.5 | |
| Magnesium | 1.5 | |
| (e) Injection I | (50 mg/ml) | |
| Compound | 5.0% | w/v |
| 1M Sodium hydroxide solution | 15.0% | v/v |
| 0.1M Hydrochloric acid (to adjust pH to 7.6) | | |
| Polyethylene glycol 400 | 4.5% | w/v |
| Water for injection to 100% | | |
| (f) Injection II | (10 mg/ml) | |
| Compound | 1.0% | W/v |
| Sodium phosphate BP | 3.6% | w/v |
| 0.1M Sodium hydroxide solution | 15.0% | v/v |
| Water for injection to 100% | | |
| (g) Injection III | (1 mg/ml, buffered to pH6) | |
| Compound | 0.1% | w/v |
| Sodium phosphate BP | 2.26% | w/v |
| Citric acid | 0.38% | w/v |
| Polyethylene glycol 400 | 3.5% | w/v |
| Water for injection to 100% | | |
| (h) Aerosol I | mg/ml | |
| Compound | 10.0 | |
| Sorbitan trioleate | 13.5 | |
| Trichlorofluoromethane | 910.0 | |
| Dichlorodifluorometha-ne | 490.0 | |
| (i) Aerosol II | mg/ml | |
| Compound | 0.2 | |
| Sorbitan trioleate | 0.27 | |
| Trichlorofluoromethane | 70.0 | |
| Dichlorodifluoromethane | 280.0 | |
| Dichlorotetrafluoroethane | 1094.0 | |
| (j) Aerosol III | mg/ml | |
| Compound | 2.5 | |
| Sorbitan trioleate | 3.38 | |
| Trichlorofluoromethane | 67.5 | |
| Dichlorodifluoromethane | 1086.0 | |
| Dichlorotetrafluoroethane | 191.6 | |
| (k) Aerosol IV | mg/ml | |
| Compound | 2.5 | |
| Soya lecithin | 2.7 | |
| Trichlorofluoromethane | 67.5 | |
| Dichlorodifluoromethane | 1086.0 | |
| Dichlorotetrafluoroethane | 191.6 | |
| (1) Ointment | ml | |
| Compound | 40 | mg |
| Ethanol | 300 | μl |
| Water | 300 | μl |
| 1-Dodecylazacycloheptan one | 50 | μl |
| Propylene glycol | to 1 | ml |

Note: These formulations may be prepared using conventional procedures well known in the pharmaceutical art. The tablets (a)-(c) may be enteric coated by conventional means, if desired to provide a coating of cellulose acetate phthalate, for example. The aerosol formulations (h)-(k) may be used in conjunction with standard, metered dose aerosol dispensers, and the suspending agents sorbitan trioleate and soya lecithin may be replaced by an alternative suspending agent such as sorbitan monooleate, sorbitan sesquioleate, polysorbate 80, polyglycerol oleate or oleic acid.

1. A compound of the formula: 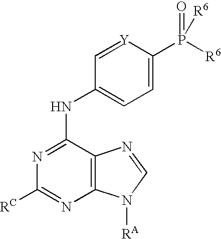
in which Y is CH or N; R^{A} is an aliphatic moiety; R⁶, in each occurrence, is an independently selected aliphatic group of 1-8 carbon atoms, or the two R⁶ moieties may be linked to one another through a carbon-carbon or carbon-heteroatom bond to form a ring, generally of up to 10 members; R^{C} is halogen, R, or -ZR, wherein Z is —O—, —S—, or NR, and each occurrence of R (or R′, R″, etc.) without a further alphanumeric superscript is independently hydrogen or an aliphatic, heteroaliphatic, aryl, or heteroaryl moiety; wherein in each of the foregoing groups, each aliphatic or heteroaliphatic moiety may be branched or unbranched, cyclic or acyclic, substituted or unsubstituted, and may contain one or more unsaturated bonds, and each aryl and heteroaryl moiety may be substituted or unsubstituted.

2. A composition comprising at least one compound of claim 1, or a pharmaceutically acceptable derivative thereof, and at least one pharmaceutically acceptable excipient or additive.

3. A method for inhibiting tumor growth and/or tumor metastasis, comprising administering a therapeutically effective amount of said compound or a pharmaceuticallly acceptable derivative thereof to a human or animal in need thereof.

4. A method for the treatment and prevention of leukemia, comprising administering a therapeutically effective amount of said compound or a pharmaceuticallly acceptable derivative thereof to a human or animal in need thereof.

5. A method for the treatment and prevention of a disease or undesirable condition which is mediated by a kinase inhibited by compound of claim 1, comprising administering a therapeutically effective amount of said compound or a pharmaceutically acceptable derivative thereof to a human or animal in need thereof.
